SEQUENCE LISTING (9 sequences)

SEQ ID NO: 1 - protein, 8 aa, Homo sapiens
DRVYIHPF

SEQ ID NO: 2 - protein, 8 aa, Artificial
  Angiotensin mimetic
DRXYXHPF

SEQ ID NO: 3 - protein, 9 aa, Homo sapiens
RPPGFSPFR

SEQ ID NO: 4 - protein, 9 aa, Artificial
  Bradykinin mimetic
RPPGXXPXR

SEQ ID NO: 5 - protein, 15 aa, Homo sapiens
GGFMTSEKSQTPLVT

SEQ ID NO: 6 - protein, 15 aa, Artificial
  Beta endorphin mimetic
GGXMXSEXSQXPLXT

SEQ ID NO: 7 - protein, 770 aa, Homo sapiens
MLPGLALLLLAAWTARALEVPTDGNAGLLAEPQIAMFCGRLNMHMNVQNGKWDSDPSGTKTCIDTKEGILQYCQEVYPELQITNVVEANQPVTIQNWCKRGRKQCKTHPHFVIPYRCLVGEFVSDALLVPDKCKFLHQERMDVCETHLHWHTVAKETCSEKSTNLHDYGMLLPCGIDKFRGVEFVCCPLAEESDNVDSADAEEDDSDVWWGGADTDYADGSEDKVVEVAEEEEVAEVEEEEADDDEDDEDGDEVEEEAEEPYEEATERTTSIATTTTTTTESVEEVVREVCSEQAETGPCRAMISRWYFDVTEGKCAPFFYGGCGGNRNNFDTEEYCMAVCGSAMSQSLLKTTQEPLARDPVKLPTTAASTPDAVDKYLETPGDENEHAHFQKAKERLEAKHRERMSQVMREWEEAERQAKNLPKADKKAVIQHFQEKVESLEQEAANERQQLVETHMARVEAMLNDRRRLALENYITALQAVPPRPRHVFNMLKKYVRAEQKDRQHTLKHFEHVRMVDPKKAAQIRSQVMTHLRVIYERMNQSLSLLYNVPAVAEEIQDEVDELLQKEQNYSDDVLANMISEPRISYGNDALMPSLTETKTTVELLPVNGEFSLDDLQPWHSFGADSVPANTENEVEPVDARPAADRGLTTRPGSGLTNIKTEEISEVKMDAEFRHDSGYEVHHQKLVFFAEDVGSNKGAIIGLMVGGVVIATVIVITLVMLKKKQYTSIHHGVVEVDAAVTPEERHLSKMQQNGYENPTYKFFEQMQN

SEQ ID NO: 8 - protein, 695 aa, Homo sapiens
MLPGLALLLLAAWTARALEVPTDGNAGLLAEPQIAMFCGRLNMHMNVQNGKWDSDPSGTKTCIDTKEGILQYCQEVYPELQITNVVEANQPVTIQNWCKRGRKQCKTHPHFVIPYRCLVGEFVSDALLVPDKCKFLHQERMDVCETHLHWHTVAKETCSEKSTNLHDYGMLLPCGIDKFRGVEFVCCPLAEESDNVDSADAEEDDSDVWWGGADTDYADGSEDKVVEVAEEEEVAEVEEEEADDDEDDEDGDEVEEEAEEPYEEATERTTSIATTTTTTTESVEEVVRVPTTAASTPDAVDKYLETPGDENEHAHFQKAKERLEAKHRERMSQVMREWEEAERQAKNLPKADKKAVIQHFQEKVESLEQEAANERQQLVETHMARVEAMLNDRRRLALENYITALQAVPPRPRHVFNMLKKYVRAEQKDRQHTLKHFEHVRMVDPKKAAQIRSQVMTHLRVIYERMNQSLSLLYNVPAVAEEIQDEVDELLQKEQNYSDDVLANMISEPRISYGNDALMPSLTETKTTVELLPVNGEFSLDDLQPWHSFGADSVPANTENEVEPVDARPAADRGLTTRPGSGLTNIKTEEISEVKMDAEFRHDSGYEVHHQKLVFFAEDVGSNKGAIIGLMVGGVVIATVIVITLVMLKKKQYTSIHHGVVEVDAAVTPEERHLSKMQQNGYENPTYKFFEQMQN

SEQ ID NO: 9 - protein, 6 aa, Artificial
  His tag
HHHHHH

(19) United States
(12) Patent Application Publication
(10) Pub. No.: US 2010/0099609 A1
(43) Pub. Date: Apr. 22, 2010

(54) eAPP AND DERIVATIVES FOR TREATMENT OF ALZHEIMER'S DISEASE

(75) Inventors: VARGHESE JOHN, San Francisco, CA (US); CLARE PETERS-LIBEU, Sebastopol, CA (US); DALE E. BREDESEN, Novato, CA (US)

Correspondence Address: Weaver Austin Villeneuve & Sampson LLP, P.O. BOX 70250, OAKLAND, CA, 94612-0250, US

(73) Assignee: BUCK INSTITUTE FOR AGE RESEARCH, Novato, CA (US)

(21) Appl. No.: 12/510,831

(22) Filed: Jul. 28, 2009

Related U.S. Application Data
(60) Provisional application No. 61/084,216, filed on Jul. 28, 2008.

(51) Int. Cl. A61K 38/16; A61K 38/10; A61P 25/28

(52) U.S. Cl. 514/12; 514/14

(57) ABSTRACT

This invention provides methods of reducing levels of amyloid beta (Aβ) protein and/or netrin-1 in a mammal. In certain embodiments the methods involve administering to the mammal a fragment of an amyloid precursor protein, or a mutant amyloid precursor protein, in an amount sufficient to decrease circulating levels of free Aβ protein in said mammal, wherein said fragment is a fragment of the extracellular domain of APP or a mutant thereof that binds amyloid beta protein and/or netrin-1.

### CROSS-REFERENCE TO RELATED APPLICATIONS

This application claims benefit of and priority to U.S. Ser. No. 61/084,216, filed on Jul. 28, 2008, which is incorporated herein by reference in its entirety for all purposes.

### STATEMENT OF GOVERNMENTAL SUPPORT

This work was supported in part by Grant Nos: NS33376 and NS45093 from the National Institutes of Health. The Government has certain rights in this invention.

### FIELD OF THE INVENTION

The present invention relates to the field of Alzheimer's disease. In particular, this invention pertains to the discovery that certain embodiments, fragments from the extracellular domain of APP 695 and/or APP770 can bind and thereby lower circulating amyloid beta protein.

### BACKGROUND OF THE INVENTION

Alzheimer's disease (AD) is a progressive degenerative disease of the brain primarily associated with aging. There also exists a hereditary form called familial Alzheimer's disease (FAD). The non-hereditary form of Alzheimer's disease, which is associated with aging, is also called sporadic Alzheimer's.

Clinical presentation of AD is characterized by loss of memory, cognition, reasoning, judgment, and orientation. As the disease progresses, motor, sensory, and linguistic abilities are also affected until there is global impairment of multiple cognitive functions. These cognitive losses occur gradually, but typically lead to severe impairment and death in the range of four to twelve years.

Alzheimer's disease is characterized by two major pathologic observations in the brain: neurofibrillary tangles (NFT) and beta amyloid (or neuritic) plaques, comprised predominantly of an aggregate of a peptide fragment known as amyloid beta (Aβ). Individuals with AD exhibit characteristic beta-amyloid deposits in the brain (beta amyloid plaques) and in cerebral blood vessels (beta amyloid angiopathy) as well as neurofibrillary tangles. Neurofibrillary tangles occur not only in Alzheimer's disease but also in other dementia-inducing disorders. On autopsy, large numbers of these lesions are generally found in areas of the human brain important for memory and cognition. Smaller numbers of these lesions in a more restricted anatomical distribution are found in the brains of most aged humans who do not have clinical AD.

Alzheimer's disease (AD) is characterized by the extracellular accumulation of amyloid plaques in the brain typically composed of the 40 or 42 amino acid amyloid beta (Aβ) peptide. This extracellular accumulation of the Aβ42 peptide is the hallmark pathology of the disease state and therefore thought to be the most important player in the cause of AD. While another common lesion of the AD brain is the presence of intracellular neurofibrillary tangles made up of abnormally phosphorylated tau, a microtubule-associated protein, currently, most evidence suggests that Aβ plays the central role in the pathogenesis of the disease. Nevertheless, the etiology of AD is still poorly understood.

The Aβ peptide is generated by endoproteolytic cleavage of the amyloid precursor protein (APP), a large type I transmembrane protein. Two enzymes that cleave APP in the amylogenic pathway are called the β- and γ-secretases which cleave APP from the N- and C-termini, respectively. In this pathway, the β-secretase (BACE) is the rate limiting enzyme in the cleavage of APP, producing as sAPP-β fragment that is secreted from the cell and a C99 fragment that is left in the membrane. The C99 fragment is the substrate for the γ-secretase (GACE) which cleaves C99 to produce Aβ and a C99 remainder that appears to function in a complex with Tip60 and Fe65 which de-repress a gene in the NFκ-B pathway through IL-1β, KAI1 (a tetraspanin cell surface molecule).

APP processing involves different secretase enzymes. For example, BACE cleavage produces sAPPβ and the C99 (or C89) fragment. The sAPPβ fragment is secreted out of the cells and C99 functions as a substrate for the γ-secretase. The γ-secretase cleaves C99 into the amyloidgenic peptides Aβ40 or Aβ42. The α-secretase cleavage produces sAPPα and C83. The sAPPα is secreted out of the cell and the C83 fragment is cleaved by the γ-secretase into the nonamyloidgenic P3 peptide.

### BRIEF SUMMARY OF THE INVENTION

In certain embodiments this invention pertains to the discovery that administration of peptide fragments from the extracellular domain of APP 695 and/or APP 770 can bind and thereby reduce levels of amyloid beta (Aβ) protein. Similarly it was discovered peptide fragments from the extracellular domain of APP 695 and/or APP 770 can bind and thereby reduce levels of nexin-1.

Accordingly, in certain embodiments, methods are provided for reducing the circulating levels of amyloid beta (Aβ) protein in a mammal. The methods typically involve administering to the mammal a fragment of an amyloid precursor protein, or a mutant amyloid precursor protein (e.g., as described herein), in an amount sufficient to decrease circulating levels of Aβ protein in said mammal, wherein said fragment is a fragment of the extracellular domain of APP or a mutant thereof that binds amyloid beta protein.

Similarly, methods are provided for reducing netrin-1 levels in a mammal. These methods typically involve administering to the mammal a fragment of an amyloid precursor protein, or a mutant amyloid precursor protein (e.g., as described herein), in an amount sufficient to decrease circulating levels of netrin-1 in said mammal, wherein said fragment is a fragment of the extracellular domain of APP or a mutant thereof that binds netrin-1.

### DEFINITIONS

The term “treat” when used with reference to treating, e.g. a pathology or disease refers to the mitigation and/or elimination of one or more symptoms of that pathology or disease, and/or a reduction in the rate of onset or severity of one or more symptoms of that pathology or disease, and/or the prevention of that pathology or disease.

The terms “isolated”, “purified”, or “biologically pure” when referring to an isolated polypeptide refer to material that is substantially or essentially free from components that normally accompany it as found in its native state. With respect to nucleic acids and/or polypeptides the term can refer to nucleic acids or polypeptides that are no longer flanked by the sequences typically flanking them in nature. Chemically synthesized polypeptides are “isolated” because they are not found in a native state (e.g. in blood, serum, etc.). In certain embodiments, the term “isolated” indicates that the polypeptide is not found in nature.

The terms “polypeptide”, “peptide” and “protein” are used interchangeably herein to refer to a polymer of amino acid residues. The terms apply to amino acid polymers in which one or more amino acid residues is an artificial chemical analogue of a corresponding naturally occurring amino acid, as well as to naturally occurring amino acid polymers. In certain embodiments the amino acid residues comprising the peptide are “L-form” amino acid residues, however, it is recognized that in various embodiments, “D” amino acids can be incorporated into the peptide. Peptides also include amino acid polymers in which one or more amino acid residues is artificial chemical analogue of a corresponding naturally occurring amino acid, as well as to naturally occurring amino acid polymers. In addition, the term applies to amino acids joined by a peptide linkage or by other, “modified linkages” (e.g., where the peptide bond is replaced by an α-ester, a β-ester, a thioamide, phosphonamide, carbomate, hydroxylate, and the like (see, e.g., Spatola, (1983) Chem. Biochem. Amino Acids and Proteins 7: 267-357), where the amide is replaced with a saturated amine (see, e.g., Skiles et al., U.S. Pat. No. 4,496,542, which is incorporated herein by reference, and Kaltenbronn et al., (1990) Pp. 969-970 in Proc. 11th American Peptide Symposium, ESCOM Science Publishers, The Netherlands, and the like)).

The term “residue” as used herein refers to natural, synthetic, or modified amino acids. Various amino acid analogues include, but are not limited to 2-aminoadipic acid, 3-aminoadipic acid, beta-alanine (beta-aminopropionic acid), 2-aminobutyric acid, 4-aminobutyric acid, piperidinic acid, 6-aminocaproic acid, 2-aminoheptanoic acid, 2-aminoisobutyric acid, 3-aminoisobutyric acid, 2-aminopimelic acid, 2,4 diaminobutyric acid, desmosine, 2,2′-diaminopimelic acid, 2,3-diaminopropionic acid, n-ethylglycine, n-ethylasparagine, hydroxylysine, allo-hydroxylysine, 3-hydroxyproline, 4-hydroxyproline, isodesmosine, allo-isoleucine, n-methylglycine, sarcosine, n-methylisoleucine, 6-n-methyllysine, n-methylvaline, norvaline, norleucine, ornithine, and the like. These modified amino acids are illustrative and not intended to be limiting.

“β-peptides” comprise of “β amino acids”, which have their amino group bonded to the β carbon rather than the α-carbon as in the 20 standard biological amino acids. The only commonly naturally occurring β amino acid is β-alanine. Where amino acid sequences are disclosed herein, amino acid sequences comprising β amino acids are also contemplated.

Peptoids, or N-substituted glycines, are a specific subclass of peptidomimetics. They are closely related to their natural peptide counterparts, but differ chemically in that their side chains are appended to nitrogen atoms along the molecule's backbone, rather than to the α-carbons (as they are in natural amino acids). Where amino acid sequences are disclosed herein, corresponding peptoids are also contemplated.

The terms “conventional” and “natural” as applied to peptides herein refer to peptides, constructed only from the naturally-occurring amino acids: Ala, Cys, Asp, Glu, Glu, Phe, Gly, His, Ile, Lys, Leu, Met, Asn, Pro, Gln, Arg, Ser, Thr, Val, Trp, and Tyr. A compound of the invention “corresponds” to a natural peptide if it elicits a biological activity (e.g., Aβ binding activity) related to the biological activity and/or specificity of the naturally occurring peptide. The elicited activity may be the same as, greater than or less than that of the natural peptide. In general, such a peptoid will have an essentially corresponding monomer sequence, where a natural amino acid is replaced by an N-substituted glycine derivative, if the N-substituted glycine derivative resembles the original amino acid in hydrophilicity, hydrophobicity, polarity, etc. Thus, for example, the following pairs of peptides would be considered “corresponding”:

Ia.

(SEQ ID NO: 1)

Asp-Arg-Val-Tyr-Ile-His-Pro-Phe (Angiotensin II)
and
Ib.

(SEQ ID NO: 2)

Asp-Arg-Val*-Tyr-Ile*-His-Pro-Phe;
IIa.

(SEQ ID NO: 3)

Arg-Pro-Pro-Gly-Phe-Ser-Pro-Phe-Arg (Bradykinin)
and
IIb:

(SEQ ID NO: 4)

Arg Pro Pro Gly Phe* Ser* Pro Phe* Arg;
IIIa:

(SEQ ID NO: 5)

Gly-Gly-Phe-Met-Thr-Ser-Glu-Lys-Ser-Gln-Thr-Pro-
Leu-Val-Thr (β-Endorphin);
and
IIIb:

(SEQ ID NO: 6)

Gly-Gly-Phe*-Met-Ser*-Ser-Glu-Lys*-Ser-Gln-Ser*-
Pro-Leu-Val*-Thr.

In these examples, “Val*” refers to N-(prop-2-yl)glycine, “Phe*” refers to N-benzylglycine, “Ser*” refers to N-(2-hydroxyethyl)glycine, “Leu*” refers to N-(2-methylprop-1-yl)glycine, and “Ile*” refers to N-(1-methylprop-1-yl)glycine. The correspondence need not be exact: for example, N-(2-hydroxyethyl)glycine may substitute for Ser, Thr, Cys, and Met;. N-(2-methylprop-1-yl)glycine may substitute for Val, Leu, and Ile. Note in IIIa and IIIb above that Ser* is used to substitute for Thr and Ser, despite the structural differences: the sidechain in Ser* is one methylene group longer than that of Ser, and differs from Thr in the site of hydroxy-substitution. In general, one may use an N-hydroxyalkyl-substituted glycine to substitute for any polar amino acid, an N-benzyl- or N-aralkyl-substituted glycine to replace any aromatic amino acid (e.g., Phe, Trp, etc.), an N-alkyl-substituted glycine such as N-butylglycine to replace any nonpolar amino acid (e.g., Leu, Val, Ile, etc.), and an N-(aminoalkyl)glycine derivative to replace any basic polar amino acid (e.g., Lys and Arg).

As used herein, “fragment” refers to a polypeptide having the sequence of at least about 9, 10, 12, 15, 18, 20, or 25 contiguous amino acids of the peptide/protein from which the fragment is “derived”. In certain embodiments, the fragment comprises at least 30, 50, 75, 100, 125, or more contiguous amino acids of the peptide/protein from which the fragment is “derived”. In this regard, it is noted that the “fragment” need not be obtained directly from the parent polypeptide, but can be synthesized de novo using chemical synthesis or recombinant expression systems.

As used herein, “APP751” and “APP770” refer, respectively, to the 751 and 770 amino acid residue long polypeptides encoded by the human APP gene (see, e.g., Selkoe (2001) Physiol Rev. 81(2): 741-766).

The term “conservative substitution” is used in reference to proteins or peptides to reflect amino acid substitutions that do not substantially alter the specificity or binding affinity (e.g., for Aβ) of the molecule. Typically conservative amino acid substitutions involve substitution one amino acid for another amino acid with similar chemical properties (e.g. charge or hydrophobicity). The following six groups each contain amino acids that are typical conservative substitutions for one another: 1) Alanine (A), Serine (S), Threonine (T); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); and 6) Phenylalanine (F), Tyrosine (Y), Tryptophan (W). Where amino acid sequences are disclosed herein, amino acid sequences comprising, one or more of the above-identified conservative substitutions are also contemplated.

### BRIEF DESCRIPTION OF THE DRAWINGS

FIG. 1 illustrates the amino acid sequence of APP 770 isoform (see, e.g., UniProtKB/Swiss-Prot entry P05067).

FIG. 2 illustrates the amino acid sequence of APP 695 isoform.

FIG. 3 illustrates the isolation of trx-eAPP from bacterial lysate.

FIGS. 4A-4C illustrate binding of LMW Aβ1-40 to eAPP. FIG. 4A: Reducing SDS PAGE analysis of selected fractions from B and C. Samples were run on 4-12% (check) NU-PAGE gel using MES running buffer after approximately 10-fold concentration. The gel was stained with Sypro Ruby. The fractions analyzed are eAPP₅₇₅₋₆₂₄+Aβ1-40 (F1), eAPP₅₇₅₋₆₂₄+Aβ1-40 (F2), eAPP₅₇₅₋₆₂₄ (F3), eAPP₁₉₋₆₂₄+Aβ1-40 (F4), eAPP₁₉₋₆₂₄ (F5). FIG. 4B: eAPP₁₉₋₆₂₄ incubated with low molecular weight Aβ1-40. The elution profiles of 150 μg eAPP₁₆₋₆₂₄ alone (•••), 150 μg eAPP₁₉₋₆₂₄ incubated with a sevenfold molar excess of low molecular weight Aβ1-40 (—) and low molecular weight Aβ1-40 alone (••—••) are compared. The elution buffer was 20 mM Tris pH 7.4, 100 mM NaCl, 2.6 mM EDTA running at 0.5 ml/min. FIG. 4C: eAPP₅₇₅₋₆₂₄ incubated with low molecular weight Aβ1-40. The elution profiles of 250 μg eAPP₅₇₅₋₆₂₄ alone (•••), 250 μg eAPP₅₇₅₋₆₂₄ incubated with a five-fold molar excess of low molecular weight Aβ1-40 (—) and low molecular weight Aβ1-40 alone (••—••) are compared. The elution buffer was 20 mM Tris pH 7.4, 125 mM NaCl, 2.6 mM EDTA running at 0.5 ml/min. For B and C, the 24 ml Superdex S200 column was calibrated in each running buffer with the High Molecular Weight Calibration Kit (Sigma). The numbers in parenthesis are the expected mass of a globular protein eluting at the indicated peak position in the appropriate running buffer. The shaded boxes indicate the fractions that were selected for reducing SDS PAGE (A) and SAXS analysis.

FIG. 5 illustrates reconstruction of the shape of eAPP₁₉₋₆₂₄ from SAXS data. Left top and bottom) Model Independent Reconstruction with the program DAMMIN. DAMMIN fits the SAXS data by determining the best arrangement of balls to fill the shape. The statistics are summarized in Table 2. For each variant 10 independent models were calculated with DAMMIN ( ) and averaged with DAMAVER ( ). Right top and bottom) BUNCH reconstruction for the eAPP. BUNCH is a modeling technique that models the protein as domains connected by a flexible chain. For all models, the domains were modeled using the x-ray crystal structures, (thioredoxin), (GFLD), Cu-binding domain) and (RERMS-CAPPD domains). Although thioredoxin was included in the models, it is not shown in the pictures in order to emphasize the similarity of the monomer with the sAPPα model. In this model, the thioredoxin domain and its flexible linker that extends outward from the GFLD domain similar to its positions in FIG. 7C. It does not interact with the rest of eAPP₁₉₋₆₂₄.

FIGS. 6A-6C illustrate the reconstruction of the shape of eAPP575-624 and its complex with LMW Aβ1-40. FIG. 6A: Model Independent Reconstruction with the program DAMMIN of eAPP₅₇₅₋₆₂₄. FIG. 6B: Model independent reconstruction of the complex of eAPP₅₇₅₋₆₂₄ and its complex with LMW Aβ1-40. FIG. 6C: BUNCH reconstruction of eAPP₅₇₅₋₆₂₄ and its complex with LMW Aβ1-40. The best reconstruction was obtained by adding one molecule of Aβ1-40 to the end of the eAPP₅₇₅₋₆₂₄. Adding more molecules of Aβ1-40 or leaving out the Aβ1-40 molecule increased the chi value of the fit to greater than 4.5. Models were calculated as in FIG. 5. The model is labeled as thioredoxin “A” and its linker “B”, Aβ cognate region “C”, Aβ1-40 “D”.

FIGS. 7A-7C show that binding of LMW Aβ1-40 competes with dimerization of eAPP19-624. FIG. 7A: Guiner plot of eAPP₁₉₋₆₂₄ (open triangle), Aβ1-40 at a molar ratio of 1:7 (filled circle), Aβ1-40 at a molar ratio of 1:10 (open circle) and Aβ1-40 at a molar ratio of 1:20 (star) normalized to concentration. The molecular mass is proportional to the extrapolated y-intercept of the line. The radius of gyration is proportional to the slope. FIG. 7B: Comparison of the DAMMIN reconstruction of eAPP₁₉₋₆₂₄ and its complex with Aβ1-40 at a molar ratio of 1:7 and at a molar ratio of 1:10. FIG. 7C: Examples of monomer models from the EOM ensemble of eAPP₁₉₋₆₂₄ and its complex with Aβ1-40. Like the eAPP₅₇₅₋₆₂₄ calculations, the best fit was obtained by adding one molecule of Aβ1-40 to the end of the eAPP₁₉₋₆₂₄. The domains are labeled as follows: Thio “A”, GFLD “B”, CuBd “C”, Acidic “D”, RERMS “E”, CAPPD “F”, Aβ cognate region “G”, Aβ1-40 “F”.

FIGS. 8A-8D illustrate binding of HMW Aβ1-40 to eAPP. FIG. 8A: eAPP₁₉₋₆₂₄ incubated with partially high molecular weight (HMW) Aβ1-40. The elution profiles of 100 μg eAPP₁₉₋₆₂₄ alone (•••), 100 μg eAPP₁₉₋₆₂₄ incubated with a sevenfold molar excess of HMW Aβ1-40 (—) and HMW Aβ1-40 alone (••—••) are compared. The elution buffer was 20 mM Tris pH 7.4, 50 mM NaCl, 2.6 mM EDTA running at 0.5 ml/min. The 24 ml Superdex 5200 column was calibrated as in FIG. 1. The shaded boxes indicate the fractions that were selected for western blot analysis with the 6E10 antibody. FIG. 8B: Cross-linking of HMW Aβ1-40 with BS3 in the same conditions in PBS. The similarity between the Stokes radius of the HMW Aβ1-40 (72 kDa) with the largest cross linked species indicate that the HMW Aβ1-40 is most likely a population of globular 12-15 mers. FIG. 8C: Western blot of eAPP19-624 cross-linked with BS3 in the presence of LMW Aβ1-40 and HMW Aβ1-40 at a 1:20 molar ratio in PBS. The blot was incubated with 6E10 at a 1:3000 dilution. FIG. 8D: Western blot of eAPP19-624 cross-linked with BS3 in the presence of LMW Aβ1-40 and HMW Aβ1-40 at a 1:20 molar ratio in PBS. The blot was incubated with 4G8 at a 1:2000 dilution. For C and D, 2 μg of eAPP₁₉₋₆₂₄ were loaded per lane.

FIGS. 9A-9C illustrate a model of LMW and HMW Aβ1-40 to eAPP₁₉₋₆₂₄. FIG. 9A: Model of LMW molecule weight Aβ1-40 drawn from PDB code 1AML (Roesch et al). FIG. 9B: Model of a unit of fibriliar Aβ1-40 drawn from PDB code 1BEG. FIG. 9C: Cartoon showing the differential binding of Aβ peptides in the helical conformation versus oligomeric Aβ peptides. In the cartoon, we have shown the APP molecules breaking into monomers upon binding of LMW Aβ1-40 as suggested by our results with the ectodomain. Residues within the transmembrane region have been shown to stabilize APP homodimers as well. Similarly, there are a variety of adaptor proteins with bind the APP cytoplasmic domain and could influence the stability of the homodimer. Therefore, it may be that efficiency of LMW Aβ1-40 in completely separating the APP homodimers depend on other factors and the LMW Aβ1-40 may be limited to destabilizing the association of the extracellular Aβ cognate regions within the homodimer. Such binding still creates kinetic opportunity for exchange of APP molecules between the homodimer and other APP containing complexes in which binding is dependant upon an exposed Aβ-cognate region.

FIG. 10 illustrates the binding of Netrin to APP.

FIGS. 11A-11D show that netrin-1 interacts with APP. APP interacts with Myc-tagged netrin-1. FIG. 11A: IP: anti-myc; FIG. 11B: IP: anti-APP. FIG. 11C: Aβ disrupts the interaction between Netrin-1 and APP. FIG. 11D, panels a-e: APP and netrin-1 colocalize in growth cones. Primary neurons of E17 embryos were stained with anti-APP (5A3/1G7 mAb) (panels a, b, c, d) and anti-netrin or with mouse and rabbit IgGs (panel e) followed by Alexa568- and Alexa488-conjugated anti-M and anti-R antibodies. Stacks of images (z step=0.25 μm) were acquired with a laser-scanning confocal microscope (Nikon PCM-2000) using a 100× objective and a 2.7 digital zoom, collected using SimplePCI (Compix Inc., OR) and processed in an SGI Octane R12 computer running Bitplane's Advanced Imaging Software. Analysis of colocalization was done using the Coloc algorithm (Imaris Bitplane). The Pearson correlation coefficient of colocalized material (c) in the region of interest was used as a measure of the degree of colocalization.

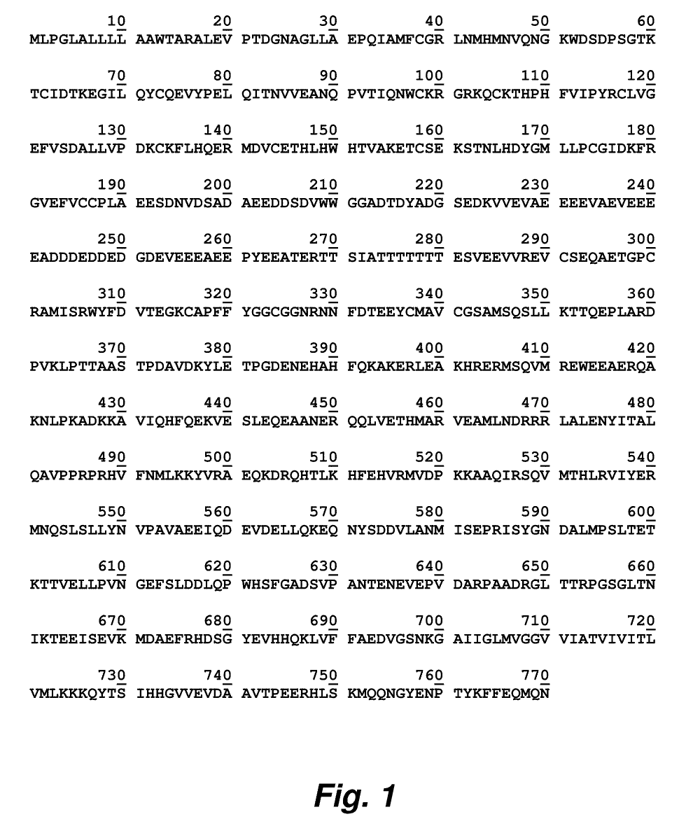

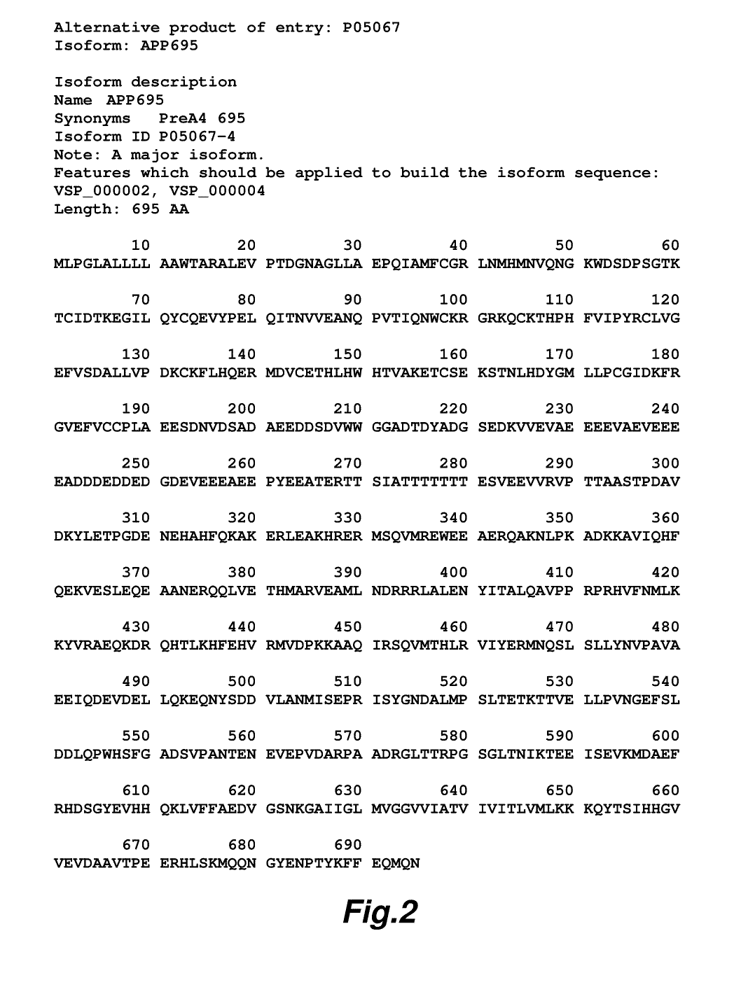

### DETAILED DESCRIPTION

This invention pertains to the discovery that the full extracellular domain of amyloid beta (Aβ) protein, or fragments, or derivatives thereof, can be administered to a mammal and, when administered in vivo, can scavenge the neurotoxic Aβ generated by neurons from the amyloid precursor protein (APP). Without being bound to a particular theory, it is believed that by forming a complex with Aβ this recombinant protein or its derivatives can remove the neurotoxic Aβ from circulation and from the brain and thus reduce the plaque load in Alzheimer's transgenic mouse models and in Alzheimer's Disease patients.

Accordingly, in certain embodiments, methods are provided for reducing the circulating levels of amyloid beta (Aβ) protein in a mammal and thereby the brain Aβ levels as well. The methods typically involve administering to the mammal (e.g., a mammal diagnosed as having or at risk for AD) a fragment of an amyloid precursor protein, or a mutant amyloid precursor protein, in an amount sufficient to decrease circulating levels of free Aβ protein in said mammal, where the fragment is a fragment of the extracellular domain of APP that binds amyloid beta (Aβ) protein or a mutant thereof that binds amyloid beta protein.

Typically, the fragment will be a fragment of, or full length extracellular domain of APP 695 and/or APP 770 that binds Aβ, a homologue thereof that binds Aβ, and/or a mutant thereof (e.g., comprising one or more conservative substitutions). In various embodiments, the fragment can comprise D amino acids, other non-natural amino acids, and/or be derivatized, e.g., to increase serum half-life. In various embodiments the fragment can be provided as a component of a pharmaceutical formulation.

In certain other embodiments, methods are provided for binding and reducing levels of netrin-1. Without being bound by a particular theory, it is noted that APP fragments bind netrin-1. Accordingly it is believed that administration of APP fragments (e.g., fragments as described herein) will result in binding to and reduction of Netrin-1 levels peripherally and thus prevent metastasis and/or tumor formation and/or growth. Free netrin is known to bind to a receptor called DCC or Unc-5 resulting in tumor formation. Thus, it is believed APP fragments can be used in the treatment of cancer and/or metastatic disease or as a prophylactic to inhibit the onset of a cancer and/or metastatic disease.

eAPP Fragments that Bind Amyloid Beta (Aβ) Protein and/or Netrin-1.

In various embodiments the peptides used in the methods of this invention include one or more fragments of the extracellular domain of APP 695 and/or APP 770 that binds Aβ and/or netrin-1. In certain embodiments the fragment comprises or consists of the amino acid sequence of at least 15 or 20 contiguous amino acids, preferably at least 25, 30, 35, or 40, more preferably at least 50, 75, or 100 contiguous amino acids from the extracellular domain of APP 695 and/or APP 770. In certain embodiments, the fragment excludes the signal sequence of the peptide (e.g., the first 17 residues of APP695).

In certain embodiments the fragment comprises or consists of the amino acid sequence of at least 15 or 20 contiguous amino acids, preferably at least 25, 30, 35, or 40, more preferably at least 50, 75, or 100 contiguous amino acids from the region of 1-624 of the APP 695 isoform, and/or from the region of residues 1-596 of the APP 695 isoform, and/or from the region of residues 499-624 of the APP 695 isoform, and/or from the region of residues 18-624 of the APP 695 isoform, and/or from residues 1-699 of the APP 770 isoform, and/or from residues 1-612 of the APP 770 isoform. In certain embodiments the fragment comprises or consists of residues 18-624 with the beginning five residues being LEVPT and the last five being EDVSNK.

In various embodiments any of these fragments can comprise one or more amino acid substitutions (mutations) replacing the native L form amino acid with a D form amino acid and/or a non-naturally occurring amino acid. Similarly, in certain embodiments, the peptide bond can be substituted with a different type of linkage as described above. In certain embodiments the mutation(s) comprise conservative substitutions. In certain embodiments the mutations comprise no more than 1, 2, 3, 4, 5, 10, 15, 20, 25, 30, 35, 40, or 50 mutations in a fragment. Where “D” amino acids are substituted for “L” amino acids, in certain embodiments all of the amino acids can be replaced with the corresponding “D” form amino acid.

In various embodiments, any of these fragments can further comprise protecting groups at the carboxyl and/or amino terminus. A wide number of protecting groups are suitable for this purpose. Such groups include, but are not limited to acetyl, amide, and alkyl groups with acetyl and alkyl groups being particularly preferred for N-terminal protection and amide groups being preferred for carboxyl terminal protection. In certain particularly preferred embodiments, the protecting groups include, but are not limited to alkyl chains as in fatty acids, propeonyl, formyl, and others. Particularly preferred carboxyl protecting groups include amides, esters, and ether-forming protecting groups. In one preferred embodiment, an acetyl group is used to protect the amino terminus and an amide group is used to protect the carboxyl terminus. These blocking groups enhance the helix-forming tendencies of the peptides. Certain particularly preferred blocking groups include alkyl groups of various lengths, e.g. groups having the formula: CH₃—(CH₂)ₙ—CO— where n ranges from about 1 to about 20, preferably from about 1 to about 16 or 18, more preferably from about 3 to about 13, and most preferably from about 3 to about 10.

In certain particularly preferred embodiments, the protecting groups include, but are not limited to alkyl chains as in fatty acids, propeonyl, formyl, and others.

Particularly preferred carboxyl protecting groups include amides, esters, and ether-forming protecting groups. In one preferred embodiment, an acetyl group is used to protect the amino terminus and/or an amide group is used to protect the carboxyl terminus. Certain particularly preferred blocking groups include alkyl groups of various lengths, e.g. groups having the formula: CH₃—(CH₂)—ₙ—CO— where n ranges from about 3 to about 20, preferably from about 3 to about 16, more preferably from about 3 to about 13, and most preferably from about 3 to about 10.

Other protecting groups include, but are not limited to Fmoc, t-butoxycarbonyl (t-BOC), 9-fluoreneacetyl group, 1-fluorenecarboxylic group, 9-florenecarboxylic group, 9-fluorenone-1-carboxylic group, benzyloxycarbonyl, Xanthyl (Xan), Trityl (Trt), 4-methyltrityl (Mtt), 4-methoxytrityl (Mmt), 4-methoxy-2,3,6-trimethyl-benzenesulphonyl (Mtr), Mesitylene-2-sulphonyl (Mts), 4,4-dimethoxybenzhydryl (Mbh), Tosyl (Tos), 2,2,5,7,8-pentamethyl chroman-6-sulphonyl (Pmc), 4-methylbenzyl (MeBzl), 4-methoxybenzyl (MeOBzl), Benzyloxy (BzlO), Benzyl (Bzl), Benzoyl (Bz), 3-nitro-2-pyridinesulphenyl (Npys), 1-(4,4-dimentyl-2,6-diaxocyclohexylidene)ethyl (Dde), 2,6-dichlorobenzyl (2,6-DiCl-Bzl), 2-chlorobenzyloxycarbonyl (2-Cl—Z), 2-bromobenzyloxycarbonyl (2-Br—Z), Benzyloxymethyl (Bom), cyclohexyloxy (cHxO), t-butoxymethyl (Bum), t-butoxy (tBuO), t-Butyl (tBu), Acetyl (Ac), and Trifluoroacetyl (TFA).

Protecting/blocking groups are well known to those of skill as are methods of coupling such groups to the appropriate residue(s) comprising the peptides of this invention (see, e.g., Greene et al., (1991) Protective Groups in Organic Synthesis, 2nd ed., John Wiley & Sons, Inc. Somerset, N.J.).

In various embodiments, any of these fragments can further comprises a tag at the N terminus. Tags (e.g., for protein isolation/purification) are well known to those of skill in the art. Suitable tags include, but are not limited to a His tag (e.g., His₆ (SEQ ID NO:7)), a FLAG tag, or a thieoredoxin protein at the N-terminus.

In various embodiments, any of the fragments described herein can be modified to bear functional groups/moieties to increase serum half-life in vivo. In certain embodiments the moieties are conjugated to the fragment.

By “conjugated” is meant the covalent linkage of at least two molecules. As described herein, in certain embodiments the peptide fragment(s) can be conjugated to a pharmaceutically acceptable polymer to increase its serum half-life. The polymer may or may not have its own biological activity. Suitable polymers include, for example, polyethylene glycol (PEG), polyvinyl pyrrolidone, polyvinyl alcohol, polyamino acids, divinylether maleic anhydride, N-(2-Hydroxypropyl)-methacrylamide, dextran, dextran derivatives including dextran sulfate, polypropylene glycol, polyoxyethylated polyol, heparin, heparin fragments, polysaccharides, cellulose and cellulose derivatives, including methylcellulose and carboxymethyl cellulose, starch and starch derivatives, polyalkylene glycol and derivatives thereof, copolymers of polyalkylene glycols and derivatives thereof, polyvinyl ethyl ethers, and .alpha.,.beta.-Poly[(2-hydroxyethyl)-DL-aspartamide, and the like, or mixtures thereof. In one preferred embodiment, the polymer is or comprises PEG (e.g., the fragment is PEGylated).

By “PEGylated” is meant the covalent attachment of at least one molecule of polyethylene glycol to the peptide fragment(s) described herein. In certain embodiments the average molecular weight of the reactant PEG is preferably between about 5,000 and about 50,000 daltons, more preferably between about 10,000 and about 40,000 daltons, and most preferably between about 15,000 and about 30,000 daltons. Particularly preferred are PEGs having nominal average sizes of about 20,000 and about 25,000 daltons. The method of attachment is not critical, but preferably does not alter, or only minimally alters, the Aβ and/or nexin-1 binding activity of the peptide. Preferably the increase in half-life is greater than any decrease in biological activity. One illustrative method of attachment is via N-terminal linkage to the polypeptide.

By “increase in serum half-life” is meant the positive change in circulating half-life of the modified peptide relative to its non-modified form. In certain embodiments serum half-life can be measured by taking blood samples at various time points after administration of the peptide and determining the concentration of that molecule in each sample. Correlation of the serum concentration with time allows calculation of the serum half-life. In various embodiments the increase in serum half-life is desirably at least about two-fold, but a smaller increase may be useful, for example where it enables a satisfactory dosing regimen or avoids a toxic effect. Preferably the increase is at least about three-fold, more preferably at least about five-fold, and most preferably at least about ten-fold, and most preferably at least about fifteen-fold.

In certain embodiments the increase in serum half-life occurs through a method that at least preserves biological activity (binding activity) of the peptide, measured, for example, in a binding assay. In some instances, the method may even increase biological activity. However, where the method does provide a decrease in biological activity, it is preferable that the proportionate increase in serum half-life is at least as great as the proportionate decrease in binding activity. More preferably, the increase in serum half-life is greater than the decrease in binding activity, proportionately. This is not an absolute requirement, and depends, for example, on the pharmacokinetics and toxicity of the specific derivative. The percentage of binding activity that is retained is preferably about 10 to about 100%, more preferably about 15 to about 100%, and most preferably about 20 to about 100%.

In various embodiments the fragment(s) described herein can be linked to a polymer through any available functionality using standard methods well known in the art. In certain embodiments it is preferred that the fragment be linked at only one position in order to minimize any disruption of its activity and to produce a pharmacologically uniform product. Nonlimiting examples of functional groups on either the polymer or biologically active molecule that can be used to form such linkages include amine and carboxy groups, thiol groups such as in cysteine resides, aldehydes and ketones, and hydroxy groups as can be found in serine, threonine, tyrosine, hydroxyproline and hydroxylysine residues.

In various embodiments the polymer can be activated by coupling a reactive group such as trichloro-s-triazine (see, e.g., Abuchowski et al. (1977) J. Biol. Chem. 252: 3582-3586), carbonylimidazole (see, e.g., Beauchamp et al. (1983) Anal. Biochem. 131: 25-33), or succinimidyl succinate (see, e.g., Abuchowski et al. (1984) Cancer Biochem. Biophys. 7:175-186) in order to react with an amine functionality on the biologically active molecule. Another coupling method involves formation of a glyoxylyl group on one molecule and an aminooxy, hydrazide or semicarbazide group on the other molecule to be conjugated (see, e.g., Fields and Dixon (1968) Biochem. J. 108: 883-887; Gaertner et al. (1992) Bioconjugate Chem. 3: 262-268; Geoghegan and Stroh (1992) Bioconjugate Chem. 3: 138-146; Gaertner et al. (1994) J. Biol. Chem. 269: 7224-7230). Other methods involve formation of an active ester at a free alcohol group of the first molecule to be conjugated using chloroformate or disuccinimidylcarbonate, which can then be conjugated to an amine group on the other molecule to be coupled (see, e.g., Veronese et al. (1985) Biochem. Biotech. 11: 141-152; Nitecki et al., U.S. Pat. No. 5,089,261; Nitecki, U.S. Pat. No. 5,281,698). Other reactive groups that can be attached via free alcohol groups are set forth in European Patent Application 0 539 167 A2, which also describes the use of imidates for coupling via free amine groups.

An aldehyde functionality useful for conjugating the fragment(s) can be generated from a functionality having adjacent amino and alcohol groups. For example, an N-terminal serine, threonine or hydroxylysine can be used to generate an aldehyde functionality via oxidative cleavage under mild conditions using periodate. These residues, or their equivalents, can be normally present, for example at the N-terminus of a polypeptide, can be exposed via chemical or enzymatic digestion, or can be introduced via recombinant or chemical methods. The reaction conditions for generating the aldehyde typically involve addition of a molar excess of sodium meta periodate and under mild conditions to avoid oxidation at other positions in the protein. The pH is preferably about 7.0. A typical reaction involves the addition of a 1.5 fold molar excess of sodium meta periodate, followed by incubation for 10 minutes at room temperature in the dark.

The aldehyde functionality can then be coupled to an activated polymer containing a hydrazide or semicarbazide functionality to form a hydrazone or semicarbazone linkage. Hydrazide-containing polymers are commercially available, and can be synthesized, if necessary, using standard techniques. PEG hydrazides for example, can be obtained from Shearwater Polymers, Inc., 2307 Spring Branch Road, Huntsville, Ala. 35801. The aldehyde is then coupled to the polymer by mixing the solution of the two components together and heating to about 37° C. until the reaction is substantially complete. An excess of the polymer hydrazide is typically used to increase the amount of conjugate obtained. A typical reaction time is 26 hours. Depending on the thermal stability of the reactants, the reaction temperature and time can be altered to provide suitable results. Detailed determination of reaction conditions for both oxidation and coupling is set forth in Geoghegan and Stroh (1992) Bioconjugate Chem. 3: 138-146).

In another embodiments, protein moieties such as the Fc fragment of human IgG can be attached to the peptides, either as chemical conjugates or expressed as fusion proteins to increase the in vivo halflife of the fragments while retaining their biological and therapeutic properties. Methods of attaching Fc and other moieties to therapeutic proteins are known to those of skill in the art (see, e.g., U.S. Patent Publication 2007/0178112.

Fragments, of the extracellular domain of APP 695 and/or APP770, homologues thereof, and/or derivatives thereof that bind amyloid beta and/or nexin-1 can be readily identified using routine screening methods. For example in an approach similar to epitope mapping, fragments of eAPP 695 and/or eAPP 770 can be assayed for their ability to bind Aβ using a gel-shift assay, a yeast two-hybrid system, a fluorescent resonance energy transfer (FRET) assay, a BIACore biding assay, and the like. With appropriate fragment selection the specific Aβ binding domain can readily be deliminted.

Similarly eAPP homologues and/or derivatives can readily be screened for their ability to bind Aβ. Such screening can be performed, for example, using a high throughput system (HTS) and literally thousands or millions of binding reactions can be screened in a day.

### Peptide Production.

The peptides described herein can be prepared by standard methods known to those of skill in the art. The peptides can, for example, be chemically synthesized, prepared from proteins, or produced using recombinant methods and techniques known in the art. Although specific techniques for their preparation are described herein, it is to be understood that all appropriate techniques suitable for production of these peptides are intended to be within the scope of this invention. Generally, these techniques include DNA and protein sequencing, cloning, expression and other recombinant engineering techniques permitting the construction of prokaryotic and eukaryotic vectors encoding and expressing each of the peptides of the invention.

Thus, for example, in certain embodiments the peptides can be chemically synthesized by any of a number of fluid or solid phase peptide synthesis techniques known to those of skill in the art. Solid phase synthesis in which the C-terminal amino acid of the sequence is attached to an insoluble support followed by sequential addition of the remaining amino acids in the sequence is a preferred method for the chemical synthesis of the polypeptides of this invention. Techniques for solid phase synthesis are well known to those of skill in the art and are described, for example, by Barany and Merrifield (1963) Solid-Phase Peptide Synthesis; pp. 3-284 in The Peptides: Analysis, Synthesis, Biology. Vol. 2: Special Methods in Peptide Synthesis, Part A.; Merrifield et al. (1963) J. Am. Chem. Soc., 85: 2149-2156, and Stewart et al. (1984) Solid Phase Peptide Synthesis, 2nd ed. Pierce Chem. Co., Rockford, Ill.

In certain embodiments the peptides of the invention may be produced by expression of a nucleic acid encoding a peptide of interest, or by cleavage from a longer length polypeptide encoded by the nucleic acid. Expression of the encoded polypeptides may be done, for example, in bacterial, yeast, plant, insect, or mammalian hosts by techniques well known in the art.

Generally recombinant expression involves creating a DNA sequence that encodes the desired peptide or fusion protein, placing the DNA in an expression cassette under the control of a particular promoter, expressing the peptide or fusion protein in a host, isolating the expressed peptide or fusion protein and, if required, renaturing the peptide or fusion protein.

DNA encoding the peptide(s) or fusion protein(s) described herein can be prepared by any suitable method as described above, including, for example, cloning and restriction of appropriate sequences or direct chemical synthesis.

This nucleic acid can be easily ligated into an appropriate vector containing appropriate expression control sequences (e.g. promoter, enhancer, etc.), and, optionally, containing one or more selectable markers (e.g. antibiotic resistance genes).

The nucleic acid sequences encoding the peptides or fusion proteins of this invention can be expressed in a variety of host cells, including, but not limited to, E. coli, other bacterial hosts, yeast, fungus, and various higher eukaryotic cells such as insect cells (e.g. SF3), the COS, CHO and HeLa cells lines and myeloma cell lines. The recombinant protein gene will typically be operably linked to appropriate expression control sequences for each host. For E. coli this can include a promoter such as the T7, trp, or lambda promoters, a ribosome binding site and preferably a transcription termination signal. For eukaryotic cells, the control sequences can include a promoter and often an enhancer (e.g., an enhancer derived from immunoglobulin genes, SV40, cytomegalovirus, etc.), and a polyadenylation sequence, and may include splice donor and acceptor sequences.

The plasmids of the invention can be transferred into the chosen host cell by well-known methods such as calcium chloride transformation for E. coli and calcium phosphate treatment or electroporation for mammalian cells. Cells transformed by the plasmids can be selected by resistance to antibiotics conferred by genes contained on the plasmids, such as the amp, gpt, neo and hyg genes.

Once expressed, the recombinant peptide(s) or fusion protein(s) can be purified according to standard procedures of the art, including ammonium sulfate precipitation, affinity columns, column chromatography, gel electrophoresis and the like (see, generally, R. Scopes, (1982) Protein Purification, Springer-Verlag, N.Y.; Deutscher (1990) Methods in Enzymology Vol. 182: Guide to Protein Purification., Academic Press, Inc. N.Y.). Substantially pure compositions of at least about 90 to 95% homogeneity are preferred, and 98 to 99% or more homogeneity are most preferred.

One of skill in the art would recognize that after chemical synthesis, biological expression, or purification, the peptide(s) or fusion protein(s) of this invention may possess a conformation substantially different than desired native conformation. In this case, it may be necessary to denature and reduce the peptide or fusion protein and then to cause the molecule to re-fold into the preferred conformation. Methods of reducing and denaturing proteins and inducing re-folding are well known to those of skill in the art (see, e.g., Debinski et al. (1993) J. Biol. Chem., 268: 14065-14070; Kreitman and Pastan (1993) Bioconjug. Chem., 4: 581-585; and Buchner, et al., (1992) Anal. Biochem., 205: 263-270). Debinski et al., for example, describes the denaturation and reduction of inclusion body proteins in guanidine-DTE. The protein is then refolded in a redox buffer containing oxidized glutathione and L-arginine.

One of skill would recognize that modifications can be made to the peptide(s) and/or fusion protein(s) proteins without diminishing their biological activity. Some modifications may be made to facilitate the cloning, expression, or incorporation of the targeting molecule into a fusion protein. Such modifications are well known to those of skill in the art and include, for example, a methionine added at the amino terminus to provide an initiation site, or additional amino acids (e.g., poly His) placed on either terminus to create conveniently located restriction sites or termination codons or purification sequences.

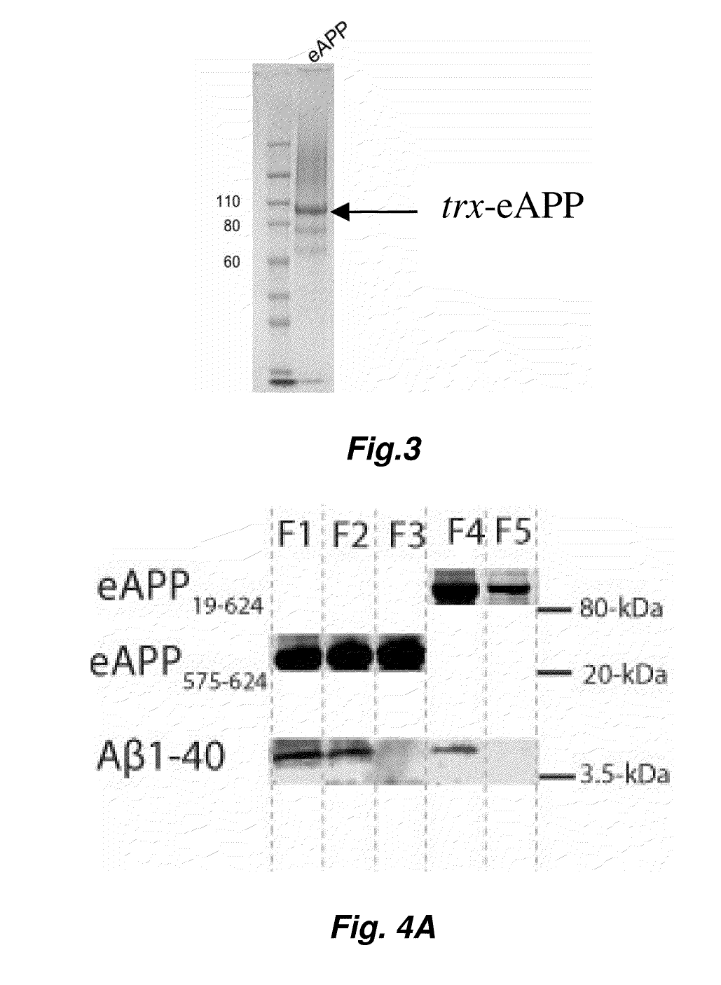

In one embodiment, the eAPP protein fragment is expressed as a thioredoxin (trx) fusion protein in pET 102/D vector (Invitrogen) in Rosetta-Gami cells (EMD Biosciences). The protein is obtained as the major product from the bacterial expression system, as seen in FIG. 3. Further purification of the protein from bacterial lysate uses IMAC chromatography followed by size exclusion chromatography. Further purification can be done on an AKTA FPLC system. Initial experiments show that trx-eAPP fragments can be produced on the large scale needed for therapy and crystallization studies and that the isolated protein is fairly stable and can be readily purified. The cleavage of the trx-eAPP is done by a protease to produce the desired fragment.

### Pharmaceutical Formulations.

In order to carry out the methods of the invention, one or more peptides of this invention are administered, e.g. to an individual diagnosed as having one or more symptoms of Alzheimer's disease, or as being at risk for Alzheimer's disease. The peptide(s) can be administered in the “native” form or, if desired, in the form of salts, esters, amides, prodrugs, derivatives, and the like, provided the salt, ester, amide, prodrug or derivative is pharmacologically suitable, i.e., effective to bind amyloid beta protein in vivo. Salts, esters, amides, prodrugs and other derivatives of the peptides can be prepared using standard procedures known to those skilled in the art of synthetic organic chemistry and described, for example, by March (1992) Advanced Organic Chemistry; Reactions, Mechanisms and Structure, 4th Ed. N.Y. Wiley-Interscience.

For example, acid addition salts are prepared from the free base using conventional methodology that typically involves reaction with a suitable acid. Generally, the base form of the drug is dissolved in a polar organic solvent such as methanol or ethanol and the acid is added thereto. The resulting salt either precipitates or can be brought out of solution by addition of a less polar solvent. Suitable acids for preparing acid addition salts include both organic acids, e.g., acetic acid, propionic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, p-toluenesulfonic acid, salicylic acid, and the like, as well as inorganic acids, e.g., hydrochloric acid, hydrobromic acid, sulfuric acid, nitric acid, phosphoric acid, and the like. An acid addition salt may be reconverted to the free base by treatment with a suitable base. Particularly preferred acid addition salts of the peptides herein are halide salts, such as may be prepared using hydrochloric or hydrobromic acids. Conversely, preparation of basic salts of the peptides of this invention are prepared in a similar manner using a pharmaceutically acceptable base such as sodium hydroxide, potassium hydroxide, ammonium hydroxide, calcium hydroxide, trimethylamine, or the like. Particularly preferred basic salts include alkali metal salts, e.g., the sodium salt, and copper salts.

Preparation of esters typically involves functionalization of hydroxyl and/or carboxyl groups which may be present within the molecular structure of the peptide. The esters are typically acyl-substituted derivatives of free alcohol groups, i.e., moieties that are derived from carboxylic acids of the formula RCOOH where R is alky, and preferably is lower alkyl. Esters can be reconverted to the free acids, if desired, by using conventional hydrogenolysis or hydrolysis procedures.

Amides and prodrugs can also be prepared using techniques known to those skilled in the art or described in the pertinent literature. For example, amides may be prepared from esters, using suitable amine reactants, or they may be prepared from an anhydride or an acid chloride by reaction with ammonia or a lower alkyl amine. Prodrugs are typically prepared by covalent attachment of a moiety that results in a compound that is therapeutically inactive until modified by an individual's metabolic system.

The peptides identified herein are useful for parenteral, topical, oral, nasal (or otherwise inhaled), rectal, or local administration, such as by aerosol or transdermally, for lowering circulating amyloid β levels and thereby also lowering brain levels of Aβ. The peptides are useful for the prophylactic and/or therapeutic treatment or prevention of one or more symptoms of Alzheimer's disease). The pharmaceutical compositions can be administered in a variety of unit dosage forms depending upon the method of administration. Suitable unit dosage forms, include, but are not limited to powders, tablets, pills, capsules, lozenges, suppositories, patches, nasal sprays, injectibles, implantable sustained-release formulations, lipid complexes, etc.

The peptides of this invention are typically combined with a pharmaceutically acceptable carrier (excipient) to form a pharmacological composition. Pharmaceutically acceptable carriers can contain one or more physiologically acceptable compound(s) that act, for example, to stabilize the composition or to increase or decrease the absorption of the peptide(s). Physiologically acceptable compounds can include, for example, carbohydrates, such as glucose, sucrose, or dextrans, antioxidants, such as ascorbic acid or glutathione, chelating agents, low molecular weight proteins, protection and uptake enhancers such as lipids, compositions that reduce the clearance or hydrolysis of the peptides, or excipients or other stabilizers and/or buffers.

Other physiologically acceptable compounds include wetting agents, emulsifying agents, dispersing agents or preservatives that are particularly useful for preventing the growth or action of microorganisms. Various preservatives are well known and include, for example, phenol and ascorbic acid. One skilled in the art would appreciate that the choice of pharmaceutically acceptable carrier(s), including a physiologically acceptable compound depends, for example, on the route of administration of the peptide(s) and on the particular physio-chemical characteristics of the peptide(s).

The excipients are preferably sterile and generally free of undesirable matter. These compositions may be sterilized by conventional, well-known sterilization techniques.

In certain applications, the compositions of this invention are administered to a mammal (e.g., to a human patient) to lower circulating levels of Aβ. In certain therapeutic applications, the compositions of this invention are administered to a patient suffering from one or more symptoms of Alzheimer's disease, or at risk for Alzheimer's disease in an amount sufficient lower ciruclating levels of Aβ and/or to prevent and/or cure and/or or at least partially prevent or arrest the disease and/or its complications. An amount adequate to accomplish this is defined as a “therapeutically effective dose.” Amounts effective for this use will depend upon the severity of the disease and the general state of the patient's health. Single or multiple administrations of the compositions may be administered depending on the dosage and frequency as required and tolerated by the patient. In any event, the composition should provide a sufficient quantity of the peptides of the formulations of this invention to effectively treat (ameliorate one or more symptoms) the patient. In certain embodiments the administration is over a period of at least one week, preferably at least two weeks, more preferably at least 3 or 4 weeks, and most preferably at least 5, 6, 7, 8, 9, 10, 11, or 12 weeks, or indefinitely.

The concentration of peptide(s) can vary widely, and will be selected primarily based on fluid volumes, viscosities, body weight and the like in accordance with the particular mode of administration selected and the patient's needs. Concentrations, however, will typically be selected to provide dosages ranging from about 0.1 or 1 mg/kg/day to about 50 mg/kg/day and sometimes higher. Typical dosages range from about 3 mg/kg/day to about 3.5 mg/kg/day, preferably from about 3.5 mg/kg/day to about 7.2 mg/kg/day, more preferably from about 7.2 mg/kg/day to about 11.0 mg/kg/day, and most preferably from about 11.0 mg/kg/day to about 15.0 mg/kg/day. In certain preferred embodiments, dosages range from about 10 mg/kg/day to about 50 mg/kg/day. In certain embodiments, dosages range from about 20 mg to about 50 mg given orally twice daily. It will be appreciated that such dosages may be varied to optimize a therapeutic regimen in a particular subject or group of subjects.

In certain preferred embodiments, the peptides of this invention are administered orally (e.g. via a tablet) or as an injectable in accordance with standard methods well known to those of skill in the art. In other preferred embodiments, the peptides may also be delivered through the skin using conventional transdermal drug delivery systems, i.e., transdermal “patches” wherein the peptide(s) are typically contained within a laminated structure that serves as a drug delivery device to be affixed to the skin. In such a structure, the drug composition is typically contained in a layer, or “reservoir,” underlying an upper backing layer. It will be appreciated that the term “reservoir” in this context refers to a quantity of “active ingredient(s)” that is ultimately available for delivery to the surface of the skin. Thus, for example, the “reservoir” may include the active ingredient(s) in an adhesive on a backing layer of the patch, or in any of a variety of different matrix formulations known to those of skill in the art. The patch may contain a single reservoir, or it may contain multiple reservoirs.

In one embodiment, the reservoir comprises a polymeric matrix of a pharmaceutically acceptable contact adhesive material that serves to affix the system to the skin during drug delivery. Examples of suitable skin contact adhesive materials include, but are not limited to, polyethylenes, polysiloxanes, polyisobutylenes, polyacrylates, polyurethanes, and the like. Alternatively, the drug-containing reservoir and skin contact adhesive are present as separate and distinct layers, with the adhesive underlying the reservoir which, in this case, may be either a polymeric matrix as described above, or it may be a liquid or hydrogel reservoir, or may take some other form. The backing layer in these laminates, which serves as the upper surface of the device, preferably functions as a primary structural element of the “patch” and provides the device with much of its flexibility. The material selected for the backing layer is preferably substantially impermeable to the peptide(s) and any other materials that are present.

In certain embodiments the peptides are administered orally. In such embodiments, peptide delivery can be enhanced by the use of protective excipients. This is typically accomplished either by complexing the polypeptide with a composition to render it resistant to acidic and enzymatic hydrolysis or by packaging the polypeptide in an appropriately resistant carrier such as a liposome. Means of protecting polypeptides for oral delivery are well known in the art (see, e.g., U.S. Pat. No. 5,391,377 describing lipid compositions for oral delivery of therapeutic agents).

In certain embodiments elevated serum half-life can be maintained by the use of sustained-release protein “packaging” systems. Such sustained release systems are well known to those of skill in the art. In one preferred embodiment, the ProLease biodegradable microsphere delivery system for proteins and peptides (Tracy (1998) Biotechnol. Prog., 14: 108; Johnson et al. (1996) Nature Med. 2: 795; Herbert et al. (1998), Pharmaceut. Res. 15, 357) a dry powder composed of biodegradable polymeric microspheres containing the peptide in a polymer matrix that can be compounded as a dry formulation with or without other agents.

The ProLease microsphere fabrication process was specifically designed to achieve a high encapsulation efficiency while maintaining integrity of the peptide. The process consists of (i) preparation of freeze-dried drug particles from bulk by spray freeze-drying the drug solution with stabilizing excipients, (ii) preparation of a drug-polymer suspension followed by sonication or homogenization to reduce the drug particle size, (iii) production of frozen drug-polymer microspheres by atomization into liquid nitrogen, (iv) extraction of the polymer solvent with ethanol, and (v) filtration and vacuum drying to produce the final dry-powder product. The resulting powder contains the solid form of the peptides, which is homogeneously and rigidly dispersed within porous polymer particles. The polymer most commonly used in the process, poly(lactide-co-glycolide) (PLG), is both biocompatible and biodegradable.

Encapsulation can be achieved at low temperatures (e.g., −40° C.). During encapsulation, the protein is maintained in the solid state in the absence of water, thus minimizing water-induced conformational mobility of the protein, preventing protein degradation reactions that include water as a reactant, and avoiding organic-aqueous interfaces where proteins may undergo denaturation. A preferred process uses solvents in which most proteins are insoluble, thus yielding high encapsulation efficiencies (e.g., greater than 95%).

In another embodiment, one or more components of the solution can be provided as a “concentrate”, e.g., in a storage container (e.g., in a premeasured volume) ready for dilution, or in a soluble capsule ready for addition to a volume of water.

The concentration of therapeutic agent in these formulations can vary widely, and will be selected primarily based on fluid volumes, viscosities, body weight and the like in accordance with the particular mode of administration selected and the patient's needs. Actual methods for preparing administrable compositions will be known or apparent to those skilled in the art and are described in more detail in such publications as Remington's Pharmaceutical Science, 15th ed., Mack Publishing Company, Easton, Pa. (1980), Remington: The Science and Practice of Pharmacy, 21st Ed. 2005, Lippincott Williams & Wilkins, and the like. The foregoing formulations and administration methods are intended to be illustrative and not limiting. It will be appreciated that, using the teaching provided herein, other suitable formulations and modes of administration can be readily devised.

Kits for Lowering Circulating Aβ Levels and/or Netrin-1 Levels.

In another embodiment this invention provides kits for lowering circulating levels of Aβ and/or for amelioration of one or more symptoms of Alzheimer's disease or for the prophylactic treatment of a subject (human or animal) at risk for Alzheimer's diseases. In certain embodiments kits are provided for lowering circulating levels of netrin-1 and/or inhibition of tumor growth and/or proliferation (e.g., in colorectal cancer).

The kits preferably comprise a container containing one or more of the peptides described herein. The peptide(s) can be provided in a unit dosage formulation (e.g. suppository, tablet, caplet, patch, etc.) and/or may be optionally combined with one or more pharmaceutically acceptable excipients.

The kit can, optionally, further comprise one or more other agents used in the treatment of the Alzheimer's disease.

In addition, the kits optionally include labeling and/or instructional materials providing directions (i.e., protocols) for the practice of the methods or use of the “therapeutics” or “prophylactics” of this invention. Preferred instructional materials describe the use of one or more peptide(s) of this invention to reduce circulating Aβ and/or netrin-1, and/or to mitigate one or more symptoms of Alzheimer's disease and/or to prevent the onset or increase of one or more of such symptoms in an individual at risk for Alzheimer's disease. The instructional materials can also, optionally, teach preferred dosages/therapeutic regiment, counter indications and the like.

While the instructional materials typically comprise written or printed materials they are not limited to such. Any medium capable of storing such instructions and communicating them to an end user is contemplated by this invention. Such media include, but are not limited to electronic storage media (e.g., magnetic discs, tapes, cartridges, chips), optical media (e.g., CD ROM), and the like. Such media may include addresses to internet sites that provide such instructional materials.

### EXAMPLES

The following examples are offered to illustrate, but not to limit the claimed invention.

### Example 1

### eAPP and Derivatives for Treatment of Alzheimer's Disease

Alzheimer's disease (AD) has been viewed largely as a disease of toxicity, mediated by the collection of a small peptide (the Aβ peptide) that damages brain cells by physical and chemical properties, such as the binding of damaging metals, reactive oxygen species production, and direct damage to cell membranes. While such effects of Aβ have been clearly demonstrated, they do not offer a physiological role for the peptide.

Our recent results indicate that Aβ has physiological signaling properties (e.g., via interaction with APP itself, the insulin-receptor, and other receptors), and our results suggest that AD may result from an imbalance between two normal processes: memory formation and normal forgetting. Our results show that APP has all of the characteristics of a dependence-receptor, i.e., a receptor that mediates cell-death in the presence of an anti-trophin (in this case, Aβ) but supports cell survival in the presence of a trophic-factor (such as laminin).

Research in our laboratory has shown that Aβ binds to APP and induces multimerization, leading to intracellular processing of APP at the caspase-site, Asp664(APP695) resulting in cellular and synaptic toxicity. As proof-of-concept we have reversed the AD-phenotype in a transgenic mouse-model by blocking the C-terminal cleavage through a single point-mutation of asp to ala (residue-664) in APP_{695(Sw,In)}. These mice lack the synapse-loss, atrophy, and electrophysiological abnormalities characteristic of PDAPP mice. We have shown that Aβ binds to its dependencereceptor APP specifically through its cognate extracellular-domain (residues: 597-624) of APP695 resulting in APP-multimerization and cell-death. Therefore, elucidating a pharmacophore-model for this interaction through 3D-structural studies facilitates the development of novel therapeutic agents for AD.

For this purpose we have recombinantly expressed and purified the full-length extracellular-domain of APP₆₉₅ (eAPP) amino acid residues 1-624, in a Rosetta-gami bacterial-expression system. We can produce milligram quantities of this recombinant protein in this expression vector. We have also developed a purification system for this protein (see, e.g., FIG. 3). We have proceeded with generating eAPP/Aβ₄₀ and eAPP/Aβ₄₂ complexes. Our experiments indicates that eAPP binds to Aβ and the structure of these complexes is under analysis using small-angle-scattering experiments (SAXS) and X-ray crystallography.

Based on these data we believe that eAPP or its derivatives when administered in vivo will scavenge the neurotoxic Aβ generated by neurons from the amyloid precursor protein (APP). We propose that by forming a complex with Aβ this recombinant protein or its homologues and/or derivatives can remove the neurotoxic Aβ from circulation and from the brain and thus reduce the plaque load in Alzheimer's transgenic mouse models and in Alzheimer's Disease patients. We therefore believe that eAPP and or its derivatives can be used as a therapy for treatment of AD.

### Example 2

### Amyloid Precursor Protein (APP)-Mediated Signal Transduction: 3D Structural Studies Toward Development Of Novel Therapeutic Agents For Alzheimer's Disease

Alzheimer's disease (AD) has been viewed largely as a disease of toxicity, mediated by the collection of a small peptide (the Aβ peptide) that damages brain cells by physical and chemical properties, such as the binding of damaging metals, reactive oxygen species production, and direct damage to cell membranes. While such effects of Aβ have been clearly demonstrated, they do not offer a physiological role for the peptide. Recent results from several different laboratories suggest Aβ has physiological signaling properties (e.g., via interaction with APP itself, the insulin-receptor, and other receptors) (Lu et al. (2003) Ann. Neurol., 54: 781-789; Ling et al. (2002) J. Neurosci., 22: 1-5; Kuner et al. (1998) J Neurosci Res, 54: 798-804), and our results suggest that AD may result from an imbalance between two normal processes: memory formation and normal forgetting. Our results show that APP has all of the characteristics of a dependence-receptor (Mehlen et al. (2994) Apoptosis, 9: 37-49), i.e., a receptor that mediates cell-death in the presence of an anti-trophin (in this case, Aβ) but supports cell survival in the presence of a trophic-factor (such as laminin).

Previous research in our laboratory has shown that Aβ binds to APP and induces multimerization, leading to intracellular processing of APP at the caspase-site, Asp664(APP695) resulting in cellular and synaptic toxicity (Saganich et al. (2006) J. Neurosci., 26: 13428-13436). As proof-of-concept we have reversed the AD-phenotype in a transgenic mouse-model by blocking the C-terminal cleavage through a single point-mutation of Asp to Ala(residue-664) in APP695(Sw,In). These mice lack the synapse-loss, atrophy, and electrophysiological abnormalities characteristic of PDAPP mice (Galvan et al. (2006) Proc. Natl. Acad. Sci., USA, 103: 7130-7135).

We have shown that Aβ binds to its dependence-receptor APP specifically through its cognate extracellular-domain (residues:597-624) of APP695 resulting in APP-multimerization and cell-death (Shaked et al. (2006) FASEB 20:1-10). Therefore, elucidating a pharmacophore-model for this interaction through 3D-structural studies would enable development of novel therapeutic agents for AD.

As a first step, we have begun to determine the 3D-structure of APP multimerized by Aβ. For this purpose we have expressed and purified the full-length extracellular-domain of APP695(eAPP) in a Rosetta-gami bacterial-expression system. Small-angle-scattering experiments (SAXS) with the eAPP protein and two other truncated homologs along with their complexes with Aβ are ongoing. We have recently obtained SAXS data on the full-length eAPP.

### BACKGROUND

APP Mediated Signal Transduction

Previously we have shown that binding of Aβ to its dependence receptor APP induces the C-terminal cleavage of APP resulting in the D664 cleavage and production of C31 (Lu et al. (2003) Ann. Neurol., 54: 781-789; Shaked et al. (2006) FASEB 20:1-10). Therapeutics that are currently being developed for AD, such as drugs that reduce β-amyloid peptide production, affect only the activity of newly formed amyloid. Similarly, therapeutics developed for disaggregation of the Aβ fibrils while preventing new amyloid deposits, do not affect the APP:Aβ binding. Thus, inhibition of APP/Aβ and its induction of C31 represent a new approach in Alzheimer's disease therapeutics

Aβ Induced Multimerization of APP

In collaborative studies with the laboratory of Prof. Edward Koo of UCSD, we found that the presence of Aβ peptide leads to multimerization of APP, inducing cleavage of APP at the caspase site, D664, and cell death.

Aβ:APP695 Complex Results in D664 Cleavage and Cell Death which is Prevented by a Single Point Mutation (D664A)

Treatment of N2a cells expressing wild type APP with Aβ results in enhanced production of the C-terminal D664 cleavage and mediates at least part of the Aβ induced cell death.

The Intracellular D664 Cleavage of APP Occurs in AD Brain.

Evidence that D664 cleavage is indeed generated in AD came from immunohistochemical analysis of AD brain using an antibody that is specific for the APP neoepitope generated on cleavage of APP. This antibody recognizes APP that is cleaved at Asp664, but does not recognize full-length APP. Reactivity was marked in the hippocampal region of patients with AD, with minimal reactivity is seen in control patients. Staining in brains from AD patients was noted intraneuronally, in some plaque and tangle-like structures and in peri-neuronal regions.

### Results.

eAPP Production and Purification.

The trx-eAPP protein (residues 18-624 of APP₆₅₆) was expressed as a thioredoxin (trx) fusion protein in pET 102/D vector (Invitrogen) in Rosetta-Gami cells (EMD Biosciences). The protein was obtained as the major product from the bacterial expression system, as seen in FIG. 3. Further purification of the protein from bacterial lysate used IMAC chromatography followed by size exclusion chromatography. Further purification can be done on an AKTA FPLC system. Initial experiments show that trx-eAPP fragments can be produced on the large scale needed for therapy and crystallization studies and that the isolated protein is fairly stable and can be readily purified. The cleavage of the trx-eAPP is done by a protease to produce the desired fragment.

X-Ray Scattering and Molecular Model.

Small angle x-ray scattering data were collected on TRX-eAPP, TRX-eAPP complex Aβ1-40 (Trx-eAPP Aβ1-40) and TRX-eAPP purified in the presence of Aβ1-20 (Trx-eAPP Aβ1-20). The center peak of each sample was concentrated to 1-5 mg/ml. Data were collected with an x-ray wavelength of 1.11 Å at Advanced Light Source beam line 12.3.1 (Advanced Light Source). Samples of the running buffer from the size exclusion columns were used for buffer subtraction. Data were processed with the program PRIMUS. The program GNOM was used to calculate the radius of gyration (67 Å for Trx-eAPP, 60 Å for Trx-eAPP Aβ1-40, 64 Å for Trx-eAPP aβ1-20) and to estimate the intensity of the scattering at zero angle which was used to estimate the molecular weight of the TRX-eAPP.

The program Bunch was used to fit the data (chi²=1.2 for Trx-eAPP, chi²=2.1 for Trx-eAPP Aβ1-40, chi²=1.7 for Trx-eAPP aβ1-20). Bunch fits the data by finding the best placement of the known crystal structures of GFLD, CuBD, RSERM, and CAPPD domains (ribbon models). Unknown regions such as acidic domain are modeled as a linked chain of dummy atoms (spheres). The best fit for each monomer of TRX-eAPP were visualized. Because SAXS at this resolution (20 Å) determines the shape of the molecule, variation of the positions of the various domains within the models may not be significant. For all three TRX-eAPP variants, the shape of the monomers were strikingly simila rot each other as well as the shape of sAPPα suggesting that the presence of Aβ1-40 does not significantly change the shape of eAPP.

Comparison with SAXS data on samples of bovine serum albumin and maltose binding protein verified that TRX-eAPP, its complex with Aβ1-40, and TRX-eAPP purified in the presence of Aβ1-20 are all dimmers in solution. For all three complexes, the calculated molecular weight was within 10% of the expected weight for a dimmer (165 kDa). In each model, the linker region and Aβ1-30 form the dimer interface. Although the shape of the eAPP is similar in the models, the angle between the monomers varies. For TRX-eAPP and TRX-eAPP purified in the presence of Aβ1-20, the angle between the monomers is close to 180°. The angle in the Aβ1-20 complex is closer to 90°. Cross-comparison of each model with the other data sets yields a chi²>7 indicating the each set of SAXS data is related to a unique conformation of TRX-eAPP dimmer.

Size-Exclusion Chromatography.

eAPP was incubated at 4° C. in 10 mM Tris pH 7.4, 150 mM NaCl, 1 mM EDTA (TBS) with a ten fold molar excess of either Aβ1-40 or Aβ1-20 in 10 mM phosphate pH 7.4, 137 mM NaCl (PBS). Partially aggregated Aβ1-40 was prepared by resuspending the lyophilized peptide in PBS and incubating at 37° C. for 48 hrs. Monomeric Aβ1-40 was prepared by dissolving he peptide in sodium hydroxide and purified by size-exclusion chromatography. Purified monomeric Aβ1-40 was prepared by dissolving the peptide in sodium hyddoxide and purified by size-exclusion chromatography. Purified monomeric Aβ1-40 was either mixed immediately with TRX-eAPP or flash frozen.

Our Data Suggest that eAPP Dimer is Parallel

Trx-eAPP(1 micromolar) was incubated with 250 micromolar BS3 in PBS for 2.5 hrs at 4° C. The reaction was then quenched with by adding 1M Tris pH 7.0 to achieve a final concentration of 50 mM. Samples were then incubated with 0.2 U Factor X for either 16 hrs at 4 deg C. or 1 hr at ° C. Samples were then analyzed by reducing SDS page and blotted with either an anti-TRX antibody or 6e10. The apparent doublet of monomeric eAPP is an artifact of the SDS page preparation, since both bands react positively with the anti-TRX and 6e10 antibodies.

The eAPP dimer exists in an open parallel conformation. IN the presence of Aβ₄₀ it orients into a partially closed conformation. This change is not seen in the presence of Aβ₁₋₂₀. We posit that the conformational change in the APP dimer induced by Aβ results in signal transduction and intracellular cleavage of APP leading to cell death.

### Example 3

### Structural Basis for Aβ Binding to App: Modulation of Processing, Signaling and Induction of Cell Death

It has been previously shown in our laboratory using cell culture models that Aβ can complex with its precursor protein APP on the cell surface and induce cell death (Shaked et al. (2006) FASEB J, 20: 1254-1256). In the presence of a single point mutation of APP695 at the Asp664 site the induction of cell death by Aβ is completely inhibited (Saganich et al. (2006) Neurosci, 26: 13428-13436; Lu et al. (2003) Ann Neurol, 54: 781-789; Lu et al. (2003) J Neurochem, 87: 733-741). Our data suggest that this interaction is similar to a ligand-receptor interaction resulting in a signal transduction event involving the intracellular cleavage of APP at the Asp-664 site, this cleavage results in the activation of a cascade of biochemical pathways resulting in cell death. In order to elucidate the structural basis for this interaction we have prepared and isolated the extracellular domain of APP₆₉₅ (residues 19-624) that we call eAPP along with two shorter fragments. We have performed SAXS (small angle x-ray scattering) analysis and obtained a low resolution structure of eAPP and of eAPP/Aβ complex.

The results of this analysis described in this example indicates a mechanism through which binding of Aβ monomers and oligomers can differentially alter APP function by binding to the ectodomain of APP and sequestering it into APP-monomers or APP-homodimers. We show that low molecular weight (LMW) Aβ 1-40, which are predominantly monomeric and in the alpha-helical conformation, binds eAPP and shifts the eAPP monomer-dimer equilibrium in favor of the monomer. Whereas high molecular weight (HMW) Aβ1-40 in the size range of Aβ*56, which are predominantly in the β-pleated horseshoe-shape conformation, bind eAPP and stabilize a dimer complex but does not alter the eAPP monomer-dimer equilibrium. Furthermore through crosslinking studies we show that the binding of Aβ to eAPP occurs at the Aβ-cognate region. Our data provides structural evidence for Aβ-APP binding and suggest a mechanistic basis on how Aβ can modulate APP processing and signaling of cell death.

### Introduction

The amyloid precursor protein (APP) which gives rise to the Aβ peptide that collects in the brains of patients with Alzheimer's disease (AD), has all of the characteristics of a dependence receptor in that it can stimulate cell death through differential ligand-binding. We have recently reported on a potential “trophic” ligand candidate for APP, the neurotrophic factor Netrin-1 that has a strong affinity for APP (Kd=5 nM) and can prevent neuronal cell death. The Aβ peptide itself can act as an “anti-trophic” ligand of APP and its binding to APP has been shown to enhance cell death (Saganich et al. (2006) Neurosci, 26: 13428-13436). APP-mediated signal transduction is implicated in the pathology of Alzheimer's Disease. Although the physiological function of APP is not completely understood, APP and its proteolytic fragments play a role in the normal making and breaking of synaptic connections in the brain. These effects are mediated both through proteolysis of APP (LaFerla et al. (2007) Nature Reviews Neuroscience 8: 499-509), but also through binding of ligands to the extracellular domain which causes intracellular signal transduction events. This argues for an entirely new view of Alzheimer's disease: currently it is thought that Alzheimer's disease is a disease of toxicity, in which the amyloid beta (Aβ) peptide damages brain cells (neurons) by physical and chemical mechanisms, such as direct membrane damage or the production of “free radicals”. However, this prevailing notion does not explain why normal cells make the Aβ peptide, nor what its normal function is. In contrast, the new hypothesis—the “dependence receptor theory of Alzheimer's disease” is that Alzheimer's disease, much like cancer, represents an imbalance in normal signaling pathways, and in this case the imbalance is between the normal making and breaking of neuronal connections. Therefore, understanding the interactions between APP and its ligands as the first step in signaling is of great interest with a view to developing novel therapeutic agents for Alzheimer's disease.

Our research has shown that the Aβ peptide and APP can behave as a signal transduction ligand receptor pair. For example, binding of Aβ1-42 to APP stimulates an intracellular cleavage of APP at residue 664 which results in cellular and synaptic toxicity. In collaborative studies with the laboratory of Prof. Edward Koo of UCSD, we found that treatment of N2a cells expressing wild type APP with Aβ1-42 leads to the formation of homo-oligomers of APP, enhanced caspase cleavage at Asp664 in the C-terminal domain and mediation of Aβ induced cell death (Lu et al. (2000) Nature Med, 6: 397-404; Lu et al. (2003) Ann Neurol, 54: 781-789; Saganich et al. (2006) Neurosci, 26: 13428-13436). Blocking the C-terminal cleavage through a single point-mutation of Asp to Ala at residue-664 in APP₆₉₅ results in the reversal of the AD-phenotype in a transgenic mouse model. These mice lack the synapse-loss, atrophy, and electrophysiological abnormalities characteristic of Alzheimer's model (PDAPP) mice even though there are very high levels of Aβ in the brain of these mice (Galvan et al. (2006) Proc. Natl. Acad. Sci., USA, 103: 7130-7135).

Aβ peptides found in the brain vary in size based on N and C-terminal proteolytic steps in the processing of APP. The most common isoform of APP in the brain is APP695 (Golde et al. (1990) Neuron 4: 253). Aβ1-42 is predominant in Alzheimer's brains (Selkoe, D. J. (1996) J. Biol. Chem. 271:18295). A high ratio of Aβ1-42 to Aβ1-40 is more predictive of the severity of AD than the overall amount of Aβ peptides or Aβ1-42. Although both Aβ1-40 and Aβ1-42 aggregate, Aβ1-42 aggregates much more quickly and has the highest propensity for forming neurotoxic Aβ oligomers which stimulate Aβ-associated cell death. While the binding of Aβ to APP has been shown to induce cell death (Shaked et al. (2006) FASEB J, 20: 1254-1256), it is unknown how the Aβ peptide or oligomer binds to APP and leads to APP-mediated signal transduction and cell death. In this study, we explored the effects of Aβ (monomer and oligomer) binding on the conformation and oligomerization state of the ectodomain of APP₆₉₅ by characterizing the complexes with biochemical techniques and small angle-x-ray scattering (SAXS).

### Methods

Protein Expression and Purification.

Fragments of the ectodomain of APP₆₉₅ were cloned into the pET-102/D-TOPO vector used a Champion Directional Cloning Kit (Invitrogen) to produce his-Patch thioredoxin fusion proteins. A stop codon was introduced after residue 624 to prevent transcription of the 6× C-terminal His-Tag. The his-Patch thioredoxin fusion proteins containing either human APP residues 19-625 (eAPP₁₉₋₆₂₄) or residues 575-625 (eAPP₅₇₅₋₆₂₄) were purified by immobilized metal ion affinity chromatography (IMAC) using a HiTrap Chelating HP column charged with nickel sulfate eluted with a 10 column volume gradient between 2 mM imidazole, 20 mM Tris pH 7.4, 100 mM NaCl to 60 mM imidazole pH 7.4. eAPP₁₉₋₆₂₄ was then loaded onto a HiTrap Heparin FF column and eluted with 10 column volume gradient between 20 mM Tris pH 7.4, 50 mM NaCl and 20 mM Tris pH 7.4, 500 mM Tris pH 7.4. eAPP₅₇₅₋₆₂₄ was loaded onto a HiTrap Q FF column and eluted with 10 column volume gradient between 20 mM Tris pH 7.4, 50 mM NaCl and 20 mM Tris pH 7.4, 500 mM Tris pH 7.4. After concentration both proteins were additionally purified by size-exclusion chromatography (HiPrep 26/60 Sephacryl S-200 column for eAPP575-624 or an HiPrep 26/60 Sephacryl S-300 column for eAPP19-624) with a running buffer of 20 mM Tris pH 7.4, 100 mM NaCl, 2.6 mM EDTA, 0.002% azide. All HiTrap and HiPrep columns were obtained from GE-Healthcare. In addition to the two thioredoxin fusion proteins, a 45-kDa C-terminal fragment of eAPP was generated during the initial steps of purification of eAPP₁₉₋₆₂₄ by an unknown protease. The C-terminal fragment was separated from eAPP₁₉₋₆₂₄ using size-exclusion chromatography.

Preparation of Aβ Peptides:

Aβ1-40, Aβ1-42, Aβ42-1 and Aβ1-20 peptides were purchased from Anaspec. Aβ1-42, Aβ42-1 and Aβ1-20 peptides were solubilized by dissolving 0.5 mg of peptide in 30 μk of 100 mM NaOH pH 11. The peptides were then diluted to 1 mg/ml using PBS (10 mM phosphate pH 7.4, 137 mM NaCl, 2.6 mM EDTA). Partially aggregated Aβ1-40 was produced by solubilizing the peptide as described and then incubating the peptides at 37° C. for 4 days in a water bath. Low molecular weight Aβ1-40 was produced by solubilizing the peptide and further purifying the solubilized peptides with size-exclusion column chromatography (Superdex 5200, GE Healthcare) using PBS as the running buffer. Aliquots of all the peptides were stored frozen at −20° C. until use.

Formation of Complexes between Aβ Peptides and eAPP Fragments

To form complexes, Aβ peptides (0.5-1.0 mg/ml) and eAPP fragments (0.05-0.1 mg/ml) were incubated for 2-3 hours on ice. The samples were then concentrated using Amicon Ultra concentrators with a 5-kDa cutoff and purified by size-exclusion column chromatography (Superdex 5200, GE Healthcare) with a running buffer of 10 mM Tris pH 7.4, 50-125 mM sodium chloride, 2.6 mM EDTA, and 0.002% (w/v) sodium azide. One to two 0.5-ml fractions were pooled from the center of the relevant peak and concentrated at 4° C. for analysis. Purity was assessed by native PAGE. All samples were stored at 4° C. for 6-12 hours before SAXS analysis.

Small-Angle Scattering Data Collection.

Small angle x-ray scattering data were collected using protein concentrations in the range of 0.1-3 mg/ml and an x-ray wavelength of 1.11 Å at beam line 12.3.1 (Advanced Light Source). Samples of the running buffer from the size-exclusion columns were used for buffer subtraction. Data were integrated with software customized for each beam line and processed with the program PRIMUS (Konarev et al. (2003) J Appl. Crystallogr 36:1277-1282). PRIMUS was used to estimate the radius of gyration and intensity at zero scattering angle for samples with concentrations less than 0.2 mg/ml as well as the Porod Volume for all samples. The program GNOM (Svergun (1992) J Appl. Crystallogr 25:495-503) was used to calculate the maximum dimension and the radius of gyration and to estimate the intensity of the scattering at zero angle for higher concentration samples. The dimensional data for each sample are summarized in Table 1. Although dilutions of each sample were analyzed to concentrations of approximately 0.1 mg/ml, no significant differences were observed in the dimensional data across the concentration ranges.

TABLE 1
SAXS Analysis of eAPP fragments and their complex with Aβ peptides.

| | | | Rg | Dmax | Porod Volume | | |
|---|---|---|---|---|---|---|---|
| Conc. | Protein:Aβ | Aβ | (Å) | (Å) | (nm³) | Oligomerization | Aβ |
| (mg/ml) | Ratio | Length | (±2) | (±5 Å) | (±10) | Number | Detected^{#} |

eAPP₁₉₋₆₂₄

| | | | | | | | |
|---|---|---|---|---|---|---|---|
| 2.8 | 1:0 | -na- | 67 | 225 | 296 | 1.9 | -na- |
| 0.58 | 1:0 | -na- | 62 | 200 | 320 | 1.6 | -na- |
| 2.0 | 1:7 | 1-20 | 65 | 210 | 310 | 2.4 | -na- |
| 0.20 | 1:20 | 42-1 | 67(±4) | 230(±10) | 294 | 2.3 | -na- |
| 0.12 | 1:20 | 1-42 | 62(±4) | 210(±10) | 269 | 2.3 | WB |
| 0.12 | 1:20 | 1-40 | 54(±4) | 160(±10) | 125 | 1.5 | SR |
| 1.2 | 1:10 | 1-40 | 51 | 160 | 145 | 1.5 | SR |
| 1.3 | 1:7 | 1-40 | 60 | 210 | 266 | 1.8 | WB |

eAPP₅₇₅₋₆₂₄

| | | | | | | | |
|---|---|---|---|---|---|---|---|
| 2.0 | 1:0 | -na- | 32 | 100 | 126 | 4.0 | -na- |
| 1.0 | 1:0 | -na- | 32 | 100 | 43 | 2.6 | -na- |
| 2.0 | 1:5 | 1-40 | 30 | 100 | 32 | 2.8 | SR |

The oligomerization number was calculated with reference to the scattering observed from proteins with known molecular weight and oligomerization state (see Supplemental Material for more detail). The estimated error in these numbers is ±20% of the calculated value which is primarily due to uncertainty in the measurement of the concentrations. To confirm the presence of Aβ in the complexes, samples were analyzed by SDS-PAGE. A band corresponding to monomeric Aβ1-40 was detected with Sypro Ruby staining (SR) or western blotting with the 4G8 antibody (WB).

The oligomerization number (O_{N}) was calculated as the ratio of the apparent mass of the protein to the expected mass derived from the protein sequence (M). For globular, non-interacting proteins, the apparent mass can be estimated by comparing the scattering of the sample to that of a reference protein (bovine serum albumin, ovalbumin, yeast alcohol dehydrogenase and carbonic anhydrase (Sigma)) with the equation

O_{N}=(C_{ref}MᵤₙIᵤₙ(0))/(CₘₒₗₗM_{ref}I_{ref}(0))

where subscripts un and ref refer to the sample and the reference protein (Svergun book). Reference proteins of different sizes were used in this study to eliminate biases due to protein size. All calculations fell into the range of ±0.2 of the number reported in Table 1 for each reference protein.

This calculation only applies to globular proteins since it assumes that all proteins have approximately the same electron density. It overestimates the apparent mass for proteins with extensive regions of random coil. Monomeric proteins with extensive random coil typically have oligomerization numbers between 1.3 and 1.8 depending on fraction of residues in the protein that adopt a random coil conformation.

### Cross-Linking Studies

Aliquots of eAPP₁₉₋₆₂₄ in PBS buffer were incubated with either low molecular weight or partially aggregated Aβ1-40 at a 1:20 molar ratio for 30 minutes at room temperature. 25 mM BS3 was added to a final concentration of 2.5 mM BS3. The final concentration for eAPP19-624 was 10 μM. The aliquots were then further incubated for 40 minutes at room temperature, before the reaction was stopped by the addition of enough 1M Tris pH 7.4 sufficient for a final concentration of 20 mM Tris pH 7.4. The samples were frozen at −20° C. prior to analysis with reducing SDS PAGE.

### Results and Discussion

To determine whether, direct binding of Aβ peptides to the ectodomain of APP₆₉₅ could modulate the oligomerization state of APP we engineered two constructs that are expressed in E. coli. The full-length construct eAPP₁₉₋₆₂₄ contains an N-terminal His-Patch thioredoxin fusion partner and the complete ectodomain of APP, referred to as eAPP, starting at the end of the signal sequence residue 19 of APP and ending at the lysine residue 624. The shorter construct, eAPP₅₇₅₋₆₂₄, is also an N-terminal thioredoxin fusion protein and contains residues 575-624 of APP₆₉₅. This region is C-terminal to the last folded domain of APP. Both these constructs end with the sequence of Aβ1-28 (Aβ cognate region). During the isolation of the full-length eAPP we also purified a C-terminal fragment of eAPP₁₉₋₆₂₄ formed through a serendipitous cleavage by an unknown E. coli protease. ESI mass spectrometry determined that the purified C-terminal fragment was a mixture of 45.0-kDa and 45.2-kD fragments starting with N-terminal residues of glu-229 and glu-230 respectively. Both the fusion proteins and the fragments are immunoreactive with the 6E10 antibody (epitope at residues 597-612) and the 4G8 antibody (epitope at residues 612-621) indicating that Aβ1-28 cognate region is fully expressed in all three proteins.

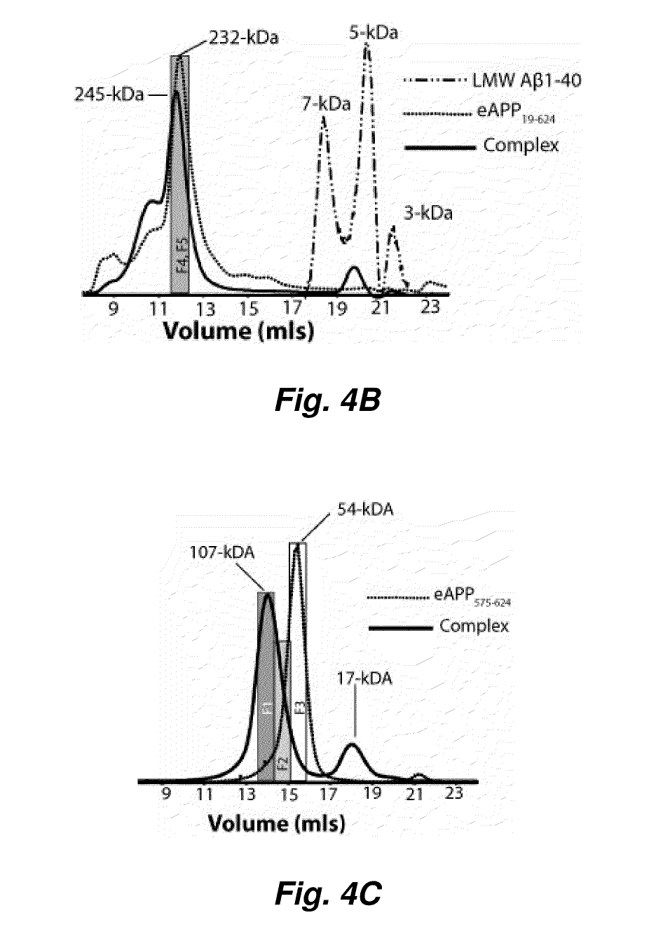

Size-exclusion chromatography (SEC) combined with SDS-PAGE, revealed that not only do Aβ1-40 peptides bind directly to fragments of the ectodomain of APP, but also confirmed our previous results in a cell based assays which indicated that at least one binding site is contained with residues 575-624 (Shaked et al. (2006) FASEB J, 20: 1254-1256). Direct visualization of the SDS-PAGE bands using Sypro Ruby or western blotting with the 6E10antibody indicate that Aβ1-40 prepared as a monomer co-elutes with the eAPP₁₉₋₆₂₄ and eAPP₅₇₅₋₆₂₄ (FIG. 4). For our analysis we prepared a low molecular weight (LMW) form of Aβ1-40 which is primarily a monomer based on SEC and a high molecular weight form (HMW) which migrates as an oligomer.

Dimerisation of the Ectodomain Through the Aβ-Cognate Region.

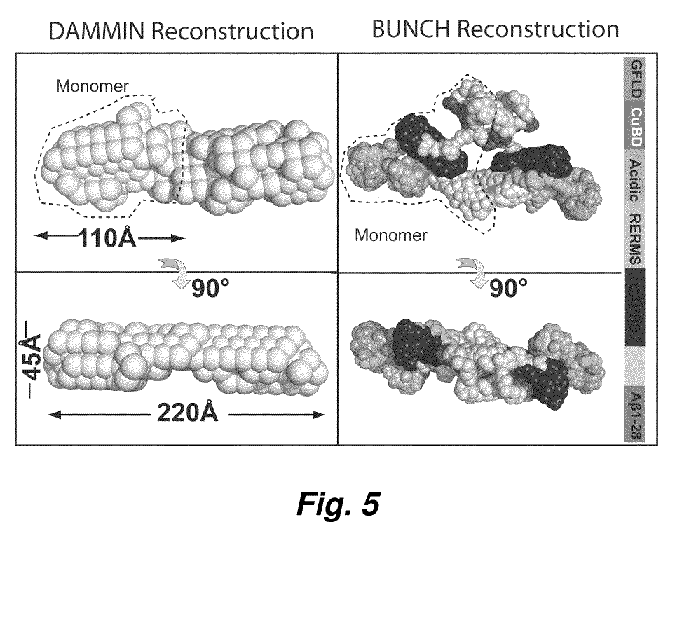

In order to determine whether Aβ-binding modulates the oligomerization state of APP, through affecting the shape or the stability of the eAPP fragments, we analyzed the eAPP fragments and their complex with low molecular weight Aβ1-40 by small angle x-ray scattering. Our results (Table 1) shows that over a wide range of concentrations (0.12-0.5 mg/ml), eAPP₁₉₋₆₂₄ was significantly larger than the monomeric sAPP_{α} construct (eAPP₁₉₋₆₁₂): radius of gyration (R_{g}) equal to 42 Å for sAPP_{α} versus 67 Å for eAPP₁₉₋₆₂₄ and maximum dimension (Dₘₐₓ) equal to 135 Å for sAPP_{α} versus 220 Å for eAPP19-624. Molecular weight calculations based on comparison of the scattering by eAPP₁₉₋₆₂₄ to the scattering of reference proteins with known molecular weight (Table 1) and shape constructions techniques such as DAMMIN (and BUNCH (Table 2) indicate eAPP₁₉₋₆₂₄ is a parallel dimer (FIG. 5). The shape of each monomer is strikingly similar to the DAMMIN reconstructions of monomeric sAPP_{α} expressed in Pichia.

TABLE 2
Modeling of eAPP₁₉₋₆₂₅ and its complex with Aβ peptides.

| Sample | | Dammin | | | |
|---|---|---|---|---|---|
| | Protein:Aβ | | | Average | χ value |
| Peptide | Ratio | Dmax | Rg | NSD (Å) | range |
| -na- | 1:0 | 219 ± 4 | 66 ± 1 | 0.988 | 1.0-1.3 |
| Aβ1-20 | 1:7 | 207 ± 2 | 64 ± 1 | 0.908 | 1.0-1.3 |
| Aβ1-40 | 1:10 | 148 ± 7 | 49 ± 2 | 0.940 | 2.6-4.7 |
| Aβ1-40 | 1:7 | 201 ± 5 | 58 ± 1 | 1.033 | 0.9-1.0 |

Sample

| | |
|---|---|
| Protein:Aβ | Best Bunch Model |

| Peptide | Ratio | Model | χ value |
|---|---|---|---|
| -na- | 1:0 | P2 Parallel | 1.2 |
| | | Dimer | |
| Aβ1-20 | 1:7 | P2 Parallel | 2.6 |
| | | Dimer | |
| Aβ1-40 | 1:10 | Monomer + | 3.6 |
| | | 2Aβ | |
| | | molecules | |
| Aβ1-40 | 1:7 | P2 Parallel | 2.0 |
| | | Dimer | |

| Sample | | EOM | | | |
|---|---|---|---|---|---|
| | Protein:Aβ | Average | | Number of | |
| Peptide | Ratio | Dₘₐₓ | Average R_{g} | Models | χ value |
| -na- | 1:0 | 152 ± 26 | 48 ± 5 | 13 | 4.6 |
| Aβ1-20 | 1:7 | 151 ± 21 | 47 ± 5 | 18 | 3.8 |
| Aβ1-40 | 1:10 | 195 ± 40 | 63 ± 14 | 17 | 1.5 |
| Aβ1-40 | 1:7 | 159 ± 26 | 49 ± 7 | 15 | 2.0 |

Dammin is a bead modeling program which calculates the shape of the molecule from the pair distribution function. The results are independent of any assumptions about numbers of residues. The reported results are derived from averaging 10 different models with Damaver. Dₘₐₓ and R_{g} are the average maximum distance and average radius of gyration respectively. The average NSD is a measure of the how well the individual models superimpose. The χ value is an assessment of how well the pair distribution function of the model matches the pair distribution function derived from the scattering data. For all three modeling methods a χ value of 1-2 indicates a very good fit for SAXS scattering from proteins. χ value's greater than 4.5 indicate that the model has no statistical relationship to the data. Bunch fits the scattering data with a mixed bead-domain model. It models the protein as a series of compact domains linked by a flexible constrained chain. The flexible chain models primarily the unstructured regions of eAPP (acidic region and the C-terminal tail), while the compact domains where modeled with the crystal structures: thioredoxin, GFLD domain, Cu binding domain, and RERMS-CAPPD domains. Although the spatial relationship between the oligomers is determined by Bunch, the symmetry and oligomerization state must be specified. For eAPP₁₉₋₆₂₅ and its complexes, the tested models included monomer, parallel dimer with or without P2 symmetry, antiparallel dimer with or without P2 symmetry and trimer models were tested. The best model and its χ value are reported in the table. EOM models the protein as compact domains linked by random coil regions. It seeks to pick an ensemble of models from 10000 randomly generated models that best fit the data. Due to limitations in the program only monomeric models could be tested.

The best-fit BUNCH model of the eAPP₁₉₋₆₂₄dimer (χ=1.5) suggests that parallel dimer is stabilized by two interfaces near the C-terminus. The major dimer P interface is composed of residues 575-624 in the C-terminal tail of eAPP. A similar interaction may stabilize full-length APP dimers within the cell membrane. Further support to stabilization of the dimer through the C-terminal interface is provided by a recently published NMR study of the C99-fragment of APP (APP₅₉₆₋₆₉₅) in a detergent bilayer found that the amides of residues in the Aβ cognate region had D₂O exchange rates typical of residues buried within a protein-protein interface. Other studies have also suggested that the Aβ-cognate region plays a critical role in formation of APP dimers through mutants and domain swapping with receptors related to APP. The assignment of the C-terminal residues to the major dimerization interface is also supported by the oligomerization state of the smaller eAPP fragments. The C-terminal proteolytic fragment (eAPP_{229/230-624}) is a dimer. Whereas, the shorter construct eAPP₅₇₅₋₆₂₄ forms a tetramer. In contrast, sAPP_{α} (eAPP₁₉₋₆₁₂) is a monomer which strongly suggests that the last 12 residues of eAPP₁₉₋₆₂₄ participate directly in the dimer interface.

A second smaller interface is located within RERMS domain. The same part of the RERMS domain participates in a homodimer interface as one crystal contacts in the RERMS-CAPPD (central APP domain) crystal structure (PDB code 1RW6). Therefore this part of eAPP has a propensity to self-associate into a dimer. Models that invert the orientation of the RERMS-CAPPD unit by stretching the acidic region had a much lower fit to the SAXS data (χ>3.5) indicating that the overall orientation of the folded domains within the dimer is correct and that it is the RERMS domain which contributes to the interface. The RERMS domain may not be the only contributor to the interface since models with near equivalent χ-values to the best-fit model (1.6<χ<2.0) place residues of the flexible acidic region in the margin of the RERMS interface. Analysis of all the fragments indicates that the interface within the RERMS region appears to prevent higher order oligomerization of APP through the Aβ cognate region as evidenced by the tetramerization of the eAPP₅₇₅₋₆₂₄. Since heparin-binding to the RERMS-CAPPD domain also stabilizes parallel dimers of sAPPα, our results suggest a mechanism through which the Aβ cognate region and components of the extracellular matrix may work together to stabilize parallel dimers of APP.

Fragments of APP containing residues 19-305 have been shown to multimerize in a redox-dependent mechanism. Similarly, association of the N-terminal domains in the context of full-length APP has been shown to promote homodimerization and potentially could affect APP's signaling properties. However, our SAXS data on full-length eAPP are inconsistent with these models in which the primary dimerization interface in the intact ectodomain lies just within the N-terminal domains. Our models of anti-parallel, or parallel dimers in which the N-terminal growth factor-like domain (GFLD) domain or the thioredoxin moiety was constrained to be in the dimer interface were inconsistent with the SAXS data (χ>4.5). Our data (FIGS. 4 and 5) suggest that eAPP forms parallel dimers through interaction at the Aβ cognate domains. We however did observe a redox-dependant oligomerisation of eAPP₁₉₋₆₂₄ after two weeks of storage at −20° C. at pH 7.4, resulting in the formation of dimers, tetramers and higher order oligomers. This oligimerisation is most likely caused by reshuffling of disulphide bonds.

LMW Aβ1-40 Binding Decreases eAPP Self-Association.

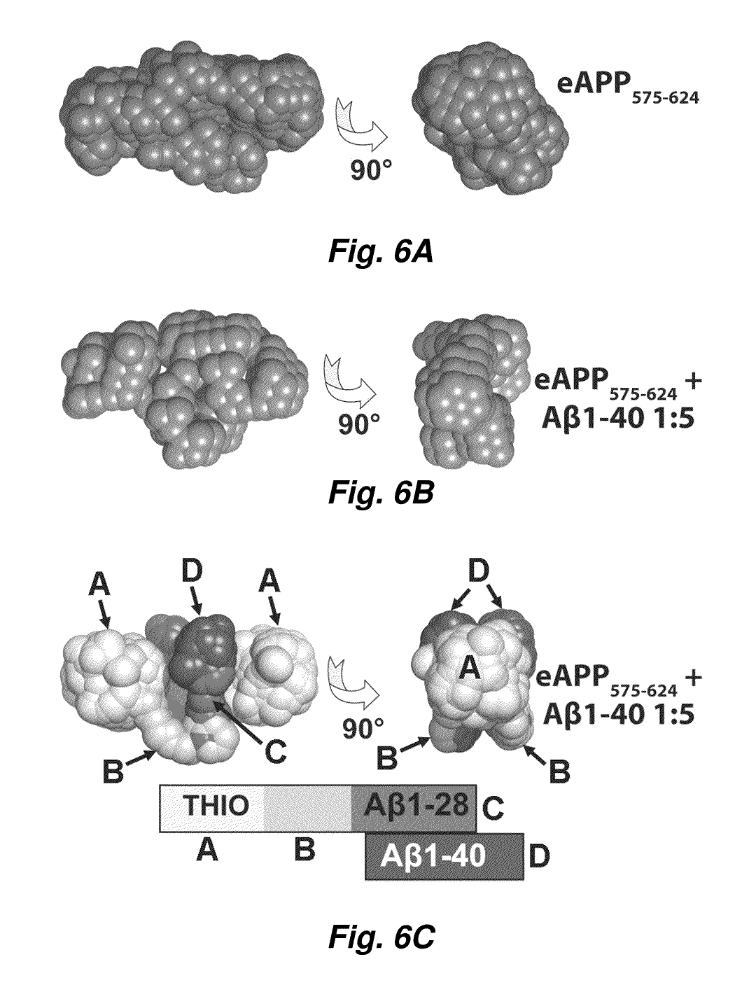

Our data shows that incubation with low-molecular weight (LMW) Aβ1-40 is sufficient to dramatically change the oligomerisation state of eAPP. For example, incubation of eAPP₅₇₅₋₆₂₄ with a five molar excess of Aβ1-40 is sufficient to reduce the average mass from a tetramer to a dimer and the Porod volume by more than two thirds (Table 1). Although the tetramer is difficult to fit unambiguously because of its globular shape (FIG. 6A), the eAPP₅₇₅₋₆₂₄ complex makes a good fit to a P2 dimer model which incorporates two Aβ1-40 molecules in the dimer interface (χ=2.4) (FIG. 6C). Models in which the thioredoxin fusion partners formed the dimer interface had a much poorer fit to the data, irrespective of whether the Aβ1-40 molecules were included in the model (χ>5).

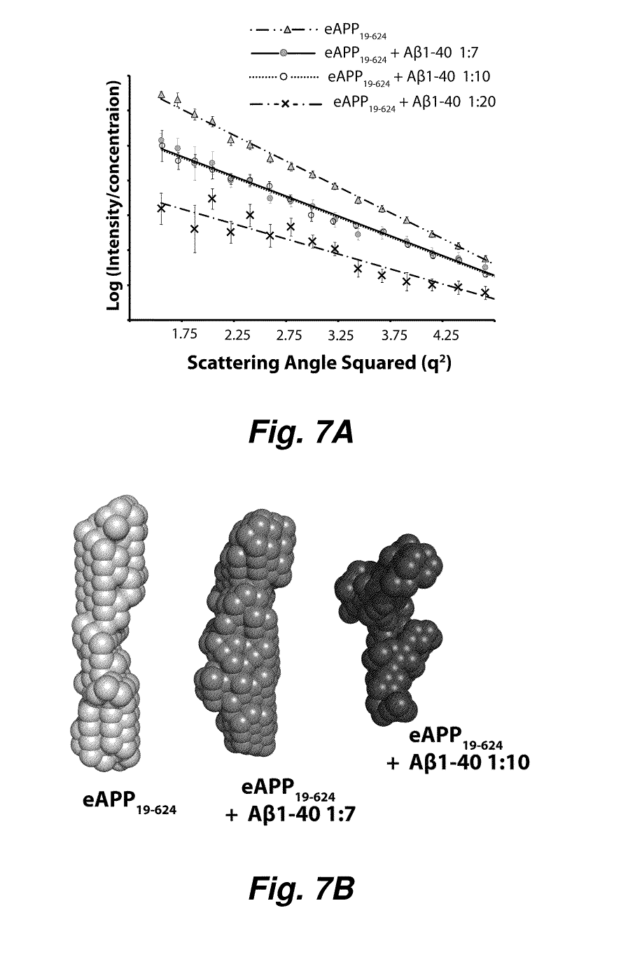

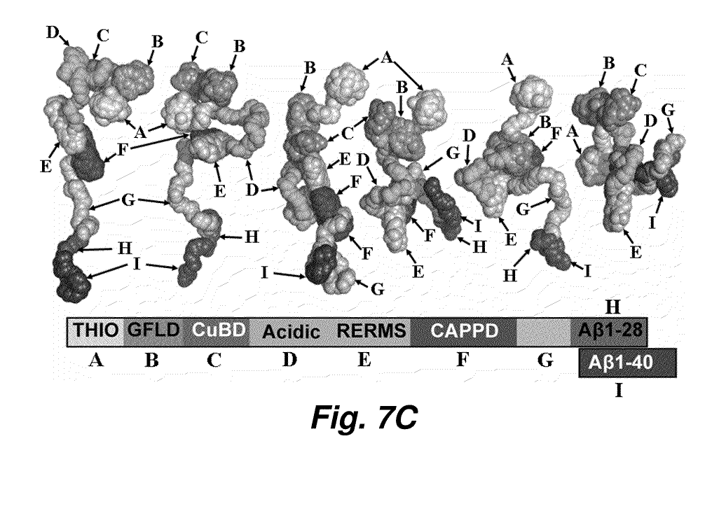

LMW Aβ1-40 also affects the oligomerization state of eAPP₁₉₋₆₂₄. Comparison of the Guinier plots shows a decrease in average molecular weight with increasing LMW Aβ1-40 (FIG. 7A). Shifting the eAPP monomer-dimer equilibrium appears to be specific to LMW Aβ1-40. Incubation of eAPP₁₉₋₆₂₄ with peptides, Aβ1-20, Aβ1-42 and Aβ42-1 (at similar molar ratios, does not significantly affect the apparent size of the eAPP₁₉₋₆₂₄ dimer (Table 1). In contrast, incubation with LMW Aβ31-40 significantly decreases the size of the eAPP₁₉₋₆₂₄ in terms of radius of gyration (R_{g}), maximum dimension (Dₘₐₓ), and Porod Volume. This decrease in volume can be best visualized by comparing the DAMMIN reconstructions for the eAPP₁₉₋₆₂₄ and eAPP₁₉₋₆₂₄ dimer incubated with Aβ1-40 at a 1:7 or 1:10 molar ratio since DAMMIN is unbiased by assumptions of shape or molecular weight. The volume of DAMMIN reconstruction from the 1:10 molar ratio incubation is nearly half that of the eAPP₁₉₋₆₂₄ dimer (FIG. 7B). The shape is in excellent agreement to the DAMMIN reconstruction for monomeric sAPPα purified from Pichia. Consistent with the interpretation that LMW Aβ1-40 binding competes with dimerization, the SAXS curve from the sample of eAPP₁₉₋₆₂₄ incubated with LMW Aβ1-40 at 1:7 molar ratio could be equally well fit as an extended monomer or a compact dimer using BUNCH suggesting that this sample may be a mixture. At the 1:10 molar incubation level, the data was best fit by treating the complex as a population of flexible monomers (FIG. 7C)—EOM analysis. This suggests that the eAPP₁₉₋₆₂₄ was not completely saturated with LMW Aβ1-40 at the 1:7 molar ratios but is a monomer at the 1:10 molar ratios based on the SAXS analysis.

Differential Interaction of LMW Aβ1-40 and HMW Aβ1-40 to the Cognate Aβ Region of eAPP

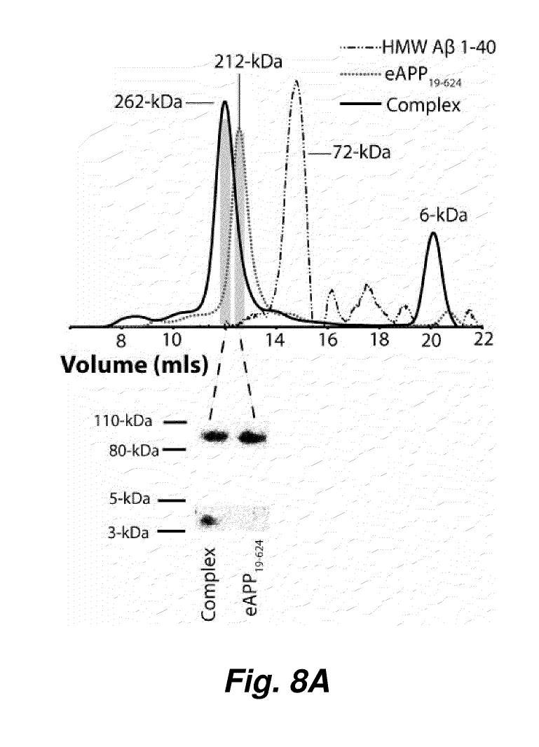

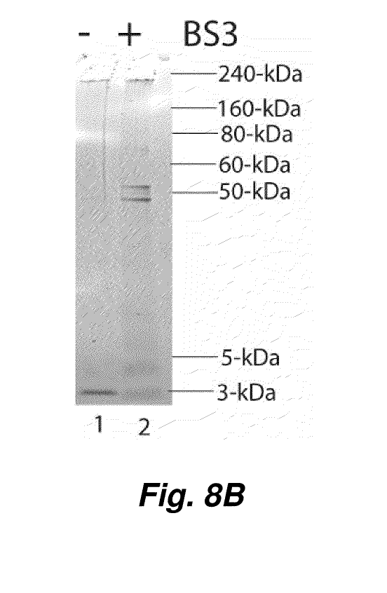

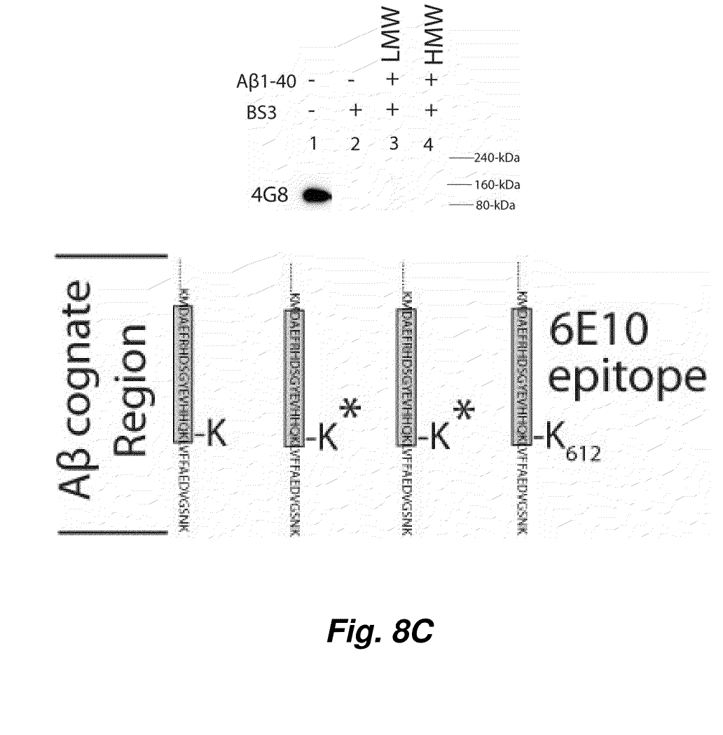

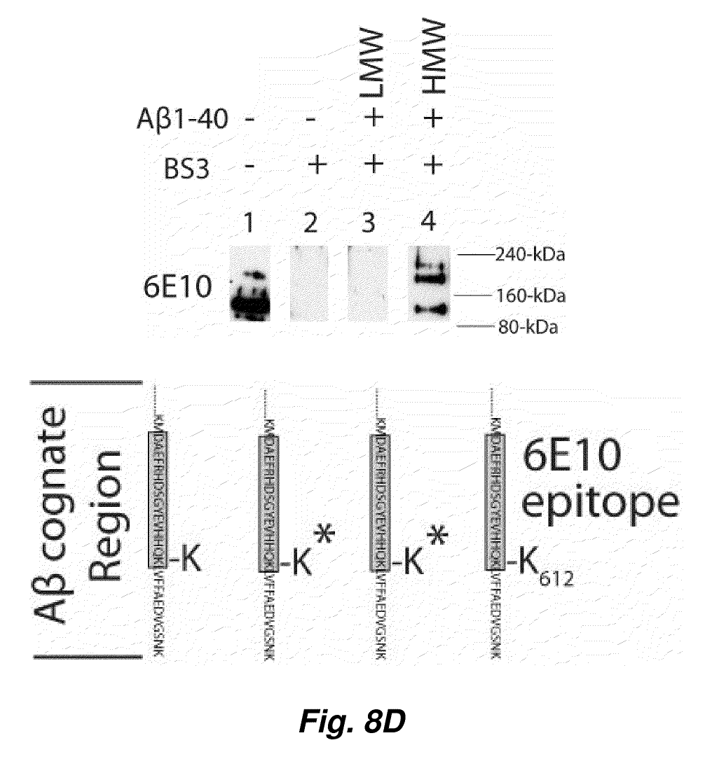

The Stokes radii of the eAPP:Aβ1-40 complex has a complex dependence on the oligomerization state of the peptides and the particular fragment of eAPP. Incubating eAPP₁₉₋₆₂₄ with high-molecular weight (HMW) Aβ1-40 oligomers (FIG. 8A) results in a species with a significantly larger Stokes radius than either component. In contrast, incubation of eAPP₁₉₋₆₂₄ with low molecular weight (LMW) Aβ1-40 results in only a subtle shift in the position of the complex peak (FIG. 4).

Using crosslinking studies we show that HMW Aβ1-40 was efficiently cross-linked by BS3 (bis[sulfosuccinimidyl] suberate) to produce two species close to 60-kDa in molecular weight (FIG. 8B) suggesting the HMW Aβ1-40 is mostly SDS-soluble oligomers formed from 12-15 Aβ1-40 molecules. That the maximum size of the oligomers is approximately 60-kDa is particularly interesting since a specific 56-kDa oligomer of Aβ (Aβ*56) has been shown to be highly neurotoxic. Although we do not know whether our oligomers contain Aβ*56, our results show that Aβ oligomers in this size range can bind eAPP₁₉₋₆₂₄.

To determine whether LMW Aβ1-40 and HMW Aβ1-40 interact similarly with the Aβ cognate region of eAPP₁₉₋₆₂₄, we conducted cross-linking experiments with these Aβ species on samples of eAPP₁₉₋₆₂₄. After incubation of eAPP₁₉₋₆₂₄ with a twenty-fold molar excess of LMW or HMW Aβ1-40 the complexes were crosslinked with a 250 fold excess of BS3. Reaction of eAPP₁₉₋₆₂₄ with such a large excess of BS3 should modify the exposed lysine residues in the protein. This reaction would also reduce the binding affinity of both 6E10 and 4G8 antibodies depending on which lysine (K-612 or K-624) within their epitope have been modified. For the lysine (K-624) in 4G8 epitope, in all cases no immunoreactivity was observed after BS3 modification suggesting that this lysine (K-624) is not protected under all incubation conditions (FIG. 8C). In contrast, the lysine (K-612) in the 6E10 epitope region was differentially protected upon incubation of eAPP₁₉₋₆₂₄ with LMW and HMW Aβ1-40. Incubation with LMW Aβ-1-40 does not protect the lysine (K-612) from crosslinking with BS3. Whereas, incubation with HMW Aβ1-40 protects this lysine (K-612) from crosslinking and as a result the 6E10 antibody can still bind (FIG. 8D). The combined results from the SAXS experiments and the crosslinking studies suggest that both LMW Aβ1-40 and HMW Aβ1-40 bind to the Aβ cognate site around the 6E10 epitope (residues 597-614) of eAPP₁₉₋₆₂₄, however only HMW Aβ1-40 affords protection of the lysine in the 6E10 epitope region. Sypro Ruby staining of these same samples indicates that the cross-linked eAPP₁₉₋₆₂₄ dimer band is present in all samples.

Our results indicate that the Aβ cognate region within the dimer bind to oligomers of Aβ (HMW Aβ1-40) in a very distinct fashion that is different from its binding to Aβ monomers in LMW Aβ1-40.

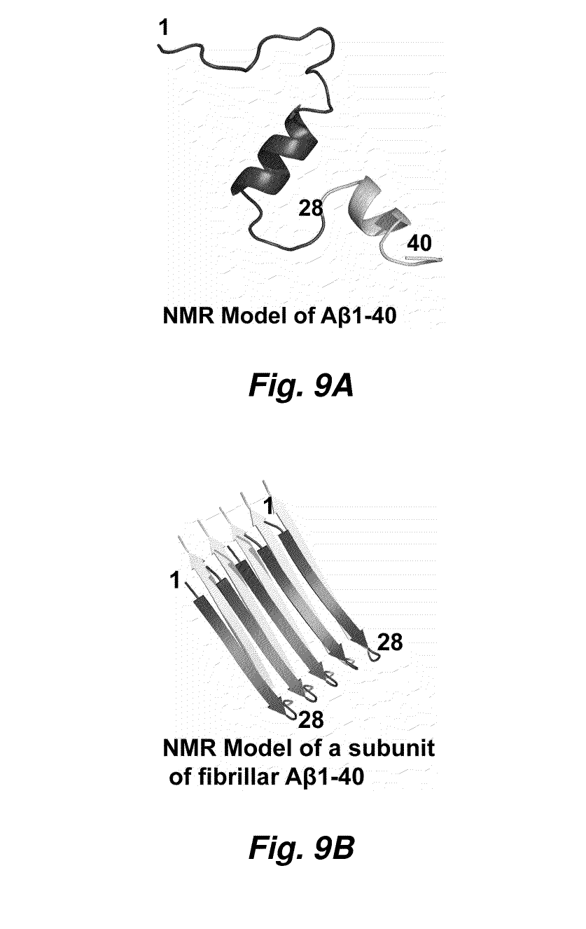

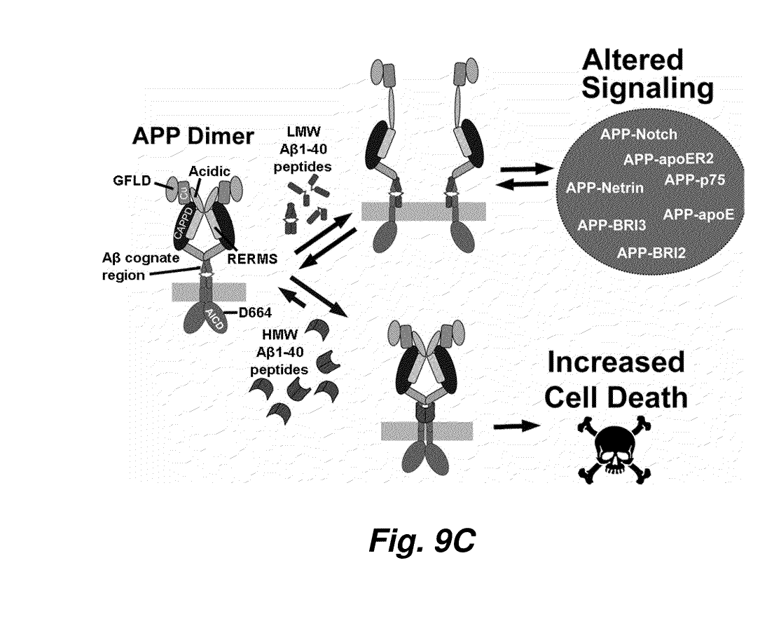

The data also suggests a mechanism through which binding of Aβ oligomers could alter APP function by sequestering APP into homodimers. The ectodomain of APP has been implicated in forming heterodimers and higher order complexes with a number of other cell surface and transmembrane proteins such as netrin, apoE, Notch, APLP1, APLP2, BRI2 and BRI3. For all of these proteins, the Aβ cognate region is required for interaction. Although the functions of the British dementia proteins, BRI2 and BRI3, are unknown, the other proteins have been implicated in signal transduction complexes that regulate the balance of neurite outgrowth or retraction. BRI3 binds to the ectodomain of APP and serves as a negative regulator of Aβ production through its interaction with BACE1. BRI2 binds to the Aβ cognate region of the C99 cleavage fragment of APP and blocks both alpha and beta-secretase activity when overexpressed. These type of interactions suggest that both the proteolytic processing of APP and the signal transduction properties of APP depend on whether APP is part of a homodimer, a heterodimer or a larger complex. Without being bound to a particular theory, we believe that one of the biological functions of LMW Aβ1-40 peptides is to destabilize APP homodimers by binding to the Aβ cognate region and assist in generating monomeric APP which could then participate in heterodimer complexes with other protein partners that can modulate APP processing and signaling (FIG. 9).

### Conclusions

Our conclusions are simple: LMW Aβ1-40 binds eAPP near the Aβ-cognate region and shifts the monomer dimer equilibrium in favor of the monomer. On the other hand, HMW Aβ1-40 in the size range of Aβ*56 also binds to eAPP dimer at the Aβ-cognate region but does not shift the monomer-dimer equilibrium. The SAXS and crosslinking experiment show that both LMW and HMW Aβ1-40 make the similar interactions with eAPP through the Aβ-cognate region but afford differential protection of the lysine (K-612) of the 6E10 epitope site. NMR studies of Aβ-peptides have previously shown that the dominant conformation of Aβ1-40 monomer such as in LMW is a helix-turn-helix motif (FIG. 9A). The LMW Aβ1-40 may be able to bind the eAPP monomer by simple substitution with the residues of the Aβ cognate region in the dimer interface and thus shift the monomer-dimer equilibrium. In contrast oligomeric Aβ in the size range of our HMW Aβ1-40 has been previously shown to have a higher proportion of (3-pleated sheet type structure such as the horseshoe-shaped fibril units (FIG. 9B). These horseshoe-shaped oligomers have two exposed Aβ1-28 sequences. Such oligomers are more likely to be able to bind two eAPP molecules at once than the helical conformation and can thus stabilize the homodimer. Therefore, we hypothesize that the effectiveness of the Aβ peptides to shift the monomer-dimer equilibrium is dependent on the relative populations of helical versus β-pleated structure in our samples (FIG. 9), and this differential binding of Aβ conformations to the ectodomain of APP can modulate APP interaction with binding partners (FIG. 9) and thus affect APP processing and signaling. Further since it is the APP homodimer that potentiates cell death (Saganich et al. (2006) Neurosci, 26: 13428-13436), stabilization of the eAPP dimer by oligomeric Aβ such as the HMW Aβ1-40 is consistent with the observations that (3-rich oligomers like Aβ*56 and fibrillar Aβ are the most efficient at inducing cell death. From a therapeutic point of view our data suggest that agents that disrupt the Aβ oligomers could shift the conformational population of Aβ from β-pleated sheet form to helix-turn-helix form resulting in differential APP binding. This in turn could effect the APP monomer-dimer equilibrium and modulate APP processing and signaling. Finally our data provides evidence at a structural level that Aβ binds the ectodomain of APP at the Aβ-cognate site, and this interaction could potentiate Aβ induced neurotoxicity seen in Alzheimer's Disease.

### Example 4

### eAPP Binds Netrin-1

In the data below we demonstrate that Netrin-1 functions as a ligand for APP.

### Recombinant Netrin-1 as a Ligand for APP.

Direct in vitro interaction between recombinant Netrin-1 and APP was assessed by immunoprecipitation and ELISA assays on recombinant βAPPs, DCC or APLP2 ecto-domain, with recombinant netrin-1 or bFGF. As revealed in the ELISA binding assay there is a specific binding interaction between Netrin-1 and APP. Both human Netrin-1 and mouse Netrin-1 bound with similar affinities to APP.

The specific binding of human Netrin-1 to βAPPs with a K_{d}˜6 nM. An

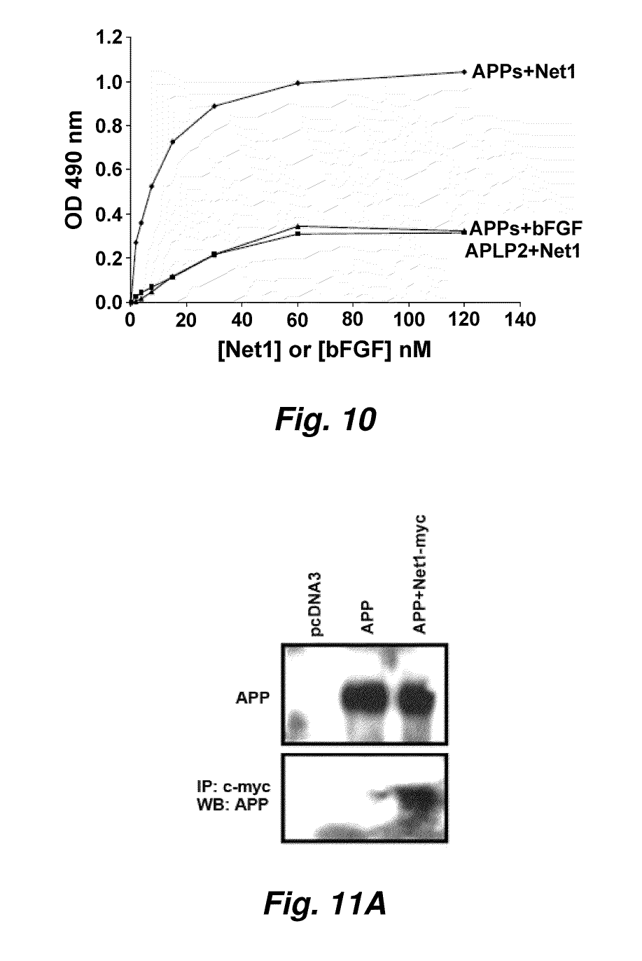

Elisa assay was developed to determine the KdAPP/netrin. 2.5 μg/ml of βAPPs protein was coated in 96-wells plate and various netrin-1 concentrations were added. Similar experiments were performed using the pair APP/bFGF or the pair APLP2/netrin-1 (see, e.g., FIG. 10). Quantification of the interaction is indicated here by the measurement of the optic density (intensity). K_{d} determination was derived from a simulated Scatchard plot (Bound/Estimated Free=f(Bound)). The affinity of Netrin-1 for APPs is the same order of magnitude as its affinity for DCC (estimated K_{dAPP/netrin} of 6 nM, compared to the known K_{dDCC/netrin-1} of 10 nM). Taken together, these data support the notion that netrin-1 interacts with APP with an affinity that is similar to that of its previously described physiological interaction with DCC.

Netrin-1 Binding Domain of APP Lies within the Aβ Region of APP.

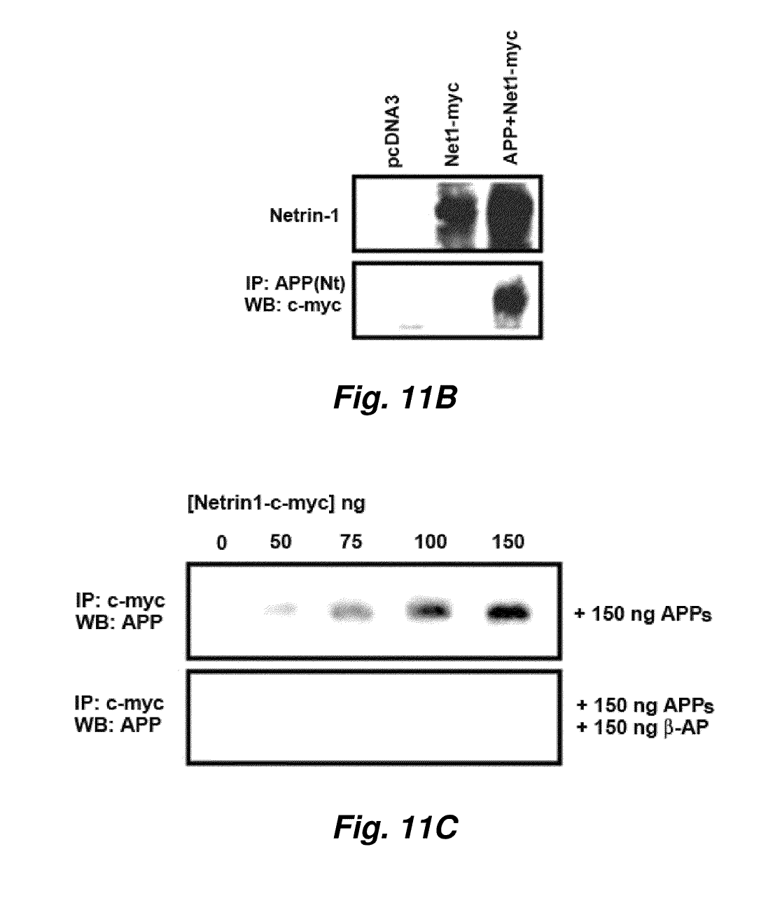

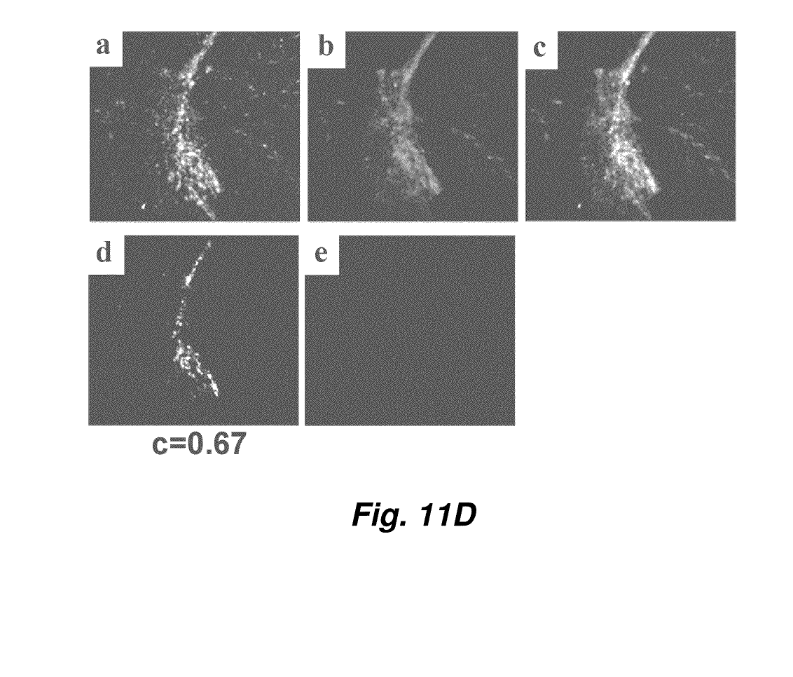

We next attempted to define the APP domain required for the APP-netrin-1 interaction. These results support the notion that APP interacts with netrin-1, and that a region of APP that corresponds to the amino-terminal portion of the Aβ peptide is sufficient for this interaction (FIG. 11). In further experiments we have demonstrated that recombinant netrin-1 also interacts in a concentration-dependent manner with the Aβ peptide. Interestingly, not only Aβ_{\tilde{4}0} but also a smaller fragment of Aβ, Aβ1-17 i.e., the 17 first amino acids of Aβ (a less toxic peptide than full-length Aβ) interacted with netrin-1, albeit with a reduced affinity (K_{dAβ/netrin-1}˜22 nM, K_{dAβ1-17/netrin-1}˜30 nM). Thus, netrin-1 interacts with a region included within the Aβ domain of APP. These, netrin-1 interaction with APP, Aβ and soluble Aβ makes it a candidate in AD therapy.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

1. A method of reducing the circulating levels of amyloid beta (Aβ) protein in a mammal, said method comprising:
administering to said mammal a fragment of an amyloid precursor protein, or a mutant amyloid precursor protein, in an amount sufficient to decrease circulating levels of free Aβ protein in said mammal, wherein said fragment is a fragment of the extracellular domain of APP or a mutant thereof that binds amyloid beta protein.

2. The method of claim 1, wherein said fragment comprises at least 15 contiguous amino acids from the region of residues 1-624 of the APP 695 isoform.

3. The method of claim 1, wherein said fragment comprises at least 15 contiguous amino acids from the region of residues 1-596 of the APP 695 isoform.

4. The method of claim 1, wherein said fragment comprises at least 15 contiguous amino acids from the region of residues 499-624 of the APP 695 isoform.

5. The method of claim 1, wherein said fragment comprises at least 15 contiguous amino acids from the region of residues 18-624 of the APP 695 isoform.

6. The method of claim 1, wherein said fragment comprises at least 15 contiguous amino acids from the eAPP fragment 575-624.

7. The method of claim 1, wherein said fragment comprises the eAPP fragment 575-624.

8. The method of claim 1, wherein said fragment comprises residues 18-624 of the APP 695 isoform.

9. The method of claim 1, wherein said fragment comprises residues 499-624 of the APP 695 isoform.

10. The method of claim 1, wherein said fragment comprises residues 18-624 with the beginning five residues being LEVPT and the last five being EDVSNK.

11. The method of claim 1, wherein said fragment comprises residues 1-624 of the APP695 isoform.

12. The method of claim 1, wherein said fragment comprises at least 15 contiguous amino acids from the region of residues 1-699 of the APP 770 isoform.

13. The method of claim 1, wherein said fragment comprises at least 15 contiguous amino acids from the region of residues 1-612 of the APP 770 isoform.

14. The method of claim 1, wherein said fragment comprises residues 1-699 of the APP 770 isoform.

15. The method of claim 1, wherein said fragment comprises no more than 10 conservative substitutions in the recited sequence.

16. The method of claim 1, wherein said fragment is a peptoid or contains β amino acids.

17. The method of claim 1, wherein said fragment comprises one or two conservative substitutions in the recited sequence.

18. The method of claim 1, wherein said fragment bears a first protecting group at the carboxyl terminus and/or a second protecting group at the amino terminus.

19. The method of claim 18, wherein said first protecting group and/or said second protecting group, when present, is independently selected from the group consisting of acetyl, amide, 3 to 20 carbon alkyl groups, Fmoc, Tboc, 9-fluoreneacetyl group, 1-fluorenecarboxylic group, 9-florenecarboxylic group, 9-fluorenone-1-carboxylic group, benzyloxycarbonyl, Xanthyl (Xan), Trityl (Trt), 4-methyltrityl (Mtt), 4-methoxytrityl (Mmt), 4-methoxy-2,3,6-trimethyl-benzenesulphonyl (Mtr), Mesitylene-2-sulphonyl (Mts), 4,4-dimethoxybenzhydryl (Mbh), Tosyl (Tos), 2,2,5,7,8-pentamethyl chroman-6-sulphonyl (Pmc), 4-methylbenzyl (MeBzl), 4-methoxybenzyl (MeOBzl), Benzyloxy (BzlO), Benzyl (Bzl), Benzoyl (Bz), 3-nitro-2-pyridinesulphenyl (Npys), 1-(4,4-dimentyl-2,6-diaxocyclohexylidene)ethyl (Dde), 2,6-dichlorobenzyl (2,6-DiCl-Bzl), 2-chlorobenzyloxycarbonyl (2-Cl—Z), 2-bromobenzyloxycarbonyl (2-Br—Z), Benzyloxymethyl (Born), t-butoxycarbonyl (Boc), cyclohexyloxy (cHxO), t-butoxymethyl (Burn), t-butoxy (tBuO), t-Butyl (tBu), and Trifluoroacetyl (TFA).

20. The method of claim 23, wherein the carboxyl terminus of said fragment is amidated.

21. The method of claim 23, wherein the amino terminus of said fragment is acetylated.

22. The method of claim 1, wherein said fragment further comprises a tag at its N-terminus.

23. The method of claim 22, wherein said tag is a tag selected from the group consisting of a His-tag, a FLAG tag, and a thieoredoxin protein.

24. The method of claim 1, wherein said fragment is derivatized with a moiety that increases serum half-life of said fragment.

25. The method of claim 24, wherein said moiety is the Fc fragment and said peptide is provided as a fusion protein with Fc.

26. The method of claim 24, wherein said moiety is selected from the group consisting of polyethylene glycol (PEG), polyvinyl pyrrolidone, polyvinyl alcohol, polyamino acid, divinylether maleic anhydride, N-(2-Hydroxypropyl)-methacrylamide, dextran, dextran derivatives, polypropylene glycol, polyoxyethylated polyol, heparin, heparin fragments, polysaccharides, cellulose, cellulose derivatives, starch, starch derivatives, dextrin, polyalkylene glycol, polyalkylene glycol derivatives, copolymers of polyalkylene glycols, polyvinyl ethyl ether, and α,β-poly[(2-hydroxyethyl)-DL-aspartamide

27. The method of claim 1, wherein said fragment is provided in a pharmaceutically acceptable excipient.

28. The method of claim 27, wherein said fragment is formulated for injection into a mammal.

29. The method of claim 27, wherein said fragment is formulated for administration by a route selected from the group consisting of oral administration, inhalation, rectal administration, intraperitoneal injection, intravascular injection, subcutaneous injection, intramuscular injection, transcutaneous administration, inhalation administration, and intramuscular injection.

30. The method of claim 1, wherein said administering is over a period of at least three weeks.

31. The method of claim 1, wherein said administering is over a period of at least six weeks.

32. The method of claim 1, wherein said mammal is a mammal diagnosed as having Alzheimer's disease or at risk for Alzheimer's disease.

33. The method of claim 1, wherein said mammal is a human diagnosed as having Alzheimer's disease.

34. A pharmaceutical formulation for reducing the circulating levels of amyloid beta (Aβ) protein and/or netrin-1 in a mammal, said formulation comprising:
a pharmaceutically acceptable excipient; and
a fragment of an amyloid precursor protein, or a mutant amyloid precursor protein, wherein said fragment is a fragment of the extracellular domain of APP that binds amyloid beta protein, or a mutant thereof that binds amyloid beta protein.

35. The formulation of claim 34, wherein said fragment comprises at least 15 contiguous amino acids from the region of residues 1-624 of the APP 695 isoform.

36. The formulation of claim 34, wherein said fragment comprises at least 15 contiguous amino acids from the region of residues 1-596 of the APP 695 isoform.

37. The formulation of claim 34, wherein said fragment comprises at least 15 contiguous amino acids from the region of residues 499-624 of the APP 695 isoform.

38. The formulation of claim 34, wherein said fragment comprises at least 15 contiguous amino acids from the region of residues 18-624 of the APP 695 isoform.

39. The formulation of claim 34, wherein said fragment comprises at least 15 contiguous amino acids from the eAPP fragment 575-624.

40. The formulation of claim 34, wherein said fragment comprises the eAPP fragment 575-624.

41. The formulation of claim 34, wherein said fragment comprises residues 18-624 of the APP 695 isoform.

42. The formulation of claim 34, wherein said fragment comprises residues 499-624 of the APP 695 isoform.

43. The formulation of claim 34, wherein said fragment comprises residues 18-624 with the beginning five residues being LEVPT and the last five being EDVSNK.

44. The formulation of claim 34, wherein said fragment comprises residues 1-624 of the APP695 isoform.

45. The formulation of claim 34, wherein said fragment comprises at least 15 contiguous amino acids from the region of residues 1-699 of the APP 770 isoform.

46. The formulation of claim 34, wherein said fragment comprises at least 15 contiguous amino acids from the region of residues 1-612 of the APP 770 isoform.

47. The formulation of claim 34, wherein said fragment comprises residues 1-699 of the APP 770 isoform.

48. The formulation of claim 34, wherein said fragment comprises at least 10 contiguous amino acids within the Aβ peptide.

49. The formulation of claim 34, wherein said fragment comprises at least 15 contiguous amino acids within the Aβ peptide.

50. The formulation of claim 34, wherein said fragment ranges in length up to 40 amino acids and comprises Aβ 1-17.

51-55. (canceled)

56. The formulation of claim 34, wherein said fragment bears a first protecting group at the carboxyl terminus and/or a second protecting group at the amino terminus.

57. The formulation of claim 56, wherein said first protecting group and/or said second protecting group, when present, is independently selected from the group consisting of acetyl, amide, 3 to 20 carbon alkyl groups, Fmoc, Tboc, 9-fluoreneacetyl group, 1-fluorenecarboxylic group, 9-florenecarboxylic group, 9-fluorenone-1-carboxylic group, benzyloxycarbonyl, Xanthyl (Xan), Trityl (Trt), 4-methyltrityl (Mtt), 4-methoxytrityl (Mmt), 4-methoxy-2,3,6-trimethyl-benzenesulphonyl (Mtr), Mesitylene-2-sulphonyl (Mts), 4,4-dimethoxybenzhydryl (Mbh), Tosyl (Tos), 2,2,5,7,8-pentamethyl chroman-6-sulphonyl (Pmc), 4-methylbenzyl (MeBzl), 4-methoxybenzyl (MeOBzl), Benzyloxy (BzlO), Benzyl (Bzl), Benzoyl (Bz), 3-nitro-2-pyridinesulphenyl (Npys), 1-(4,4-dimentyl-2,6-diaxocyclohexylidene)ethyl (Dde), 2,6-dichlorobenzyl (2,6-DiCl-Bzl), 2-chlorobenzyloxycarbonyl (2-Cl—Z), 2-bromobenzyloxycarbonyl (2-Br—Z), Benzyloxymethyl (Bom), t-butoxycarbonyl (Boc), cyclohexyloxy (cHxO), t-butoxymethyl (Bum), t-butoxy (tBuO), t-Butyl (tBu), and Trifluoroacetyl (TFA).

58. The formulation of claim 56, wherein the carboxyl terminus of said fragment is amidated.

59. The formulation of claim 58, wherein the carboxyl terminus of said fragment is acetylated.

60-66. (canceled)

67. A kit for reducing the circulating levels of amyloid beta (Aβ) protein in a mammal, said kit comprising:
a container containing a fragment of an amyloid precursor protein, or a mutant amyloid precursor protein, wherein said fragment is a fragment of the extracellular domain of APP that binds amyloid beta protein, or a mutant thereof that binds amyloid beta protein; and
instructional materials teaching the use of said fragment for reducing circulating levels of amyloid beta protein in a mammal.

68. A method of reducing netrin-1 levels in a mammal, said method comprising administering to said mammal a fragment of an amyloid precursor protein, or a mutant amyloid precursor protein, in an amount sufficient to decrease circulating levels of netrin-1 in said mammal, wherein said fragment is a fragment of the extracellular domain of APP or a mutant thereof that binds netrin-1.

69-70. (canceled)

71. The method of claim 68, wherein administering comprises administering said fragment wherein said fragment ranges in length up to 40 amino acids and comprises Aβ1-17.

72-100. (canceled)

101. The method of claim 71, wherein said fragment is formulated for administration by a route selected from the group consisting of oral administration, inhalation, rectal administration, intraperitoneal injection, intravascular injection, subcutaneous injection, intramuscular injection, transcutaneous administration, inhalation administration, and intramuscular injection.

102. The method of claim 71, wherein said administering is over a period of at least three weeks.

103-106. (canceled)

107. A method of mitigating one or more symptoms of Alzheimer's disease, said method comprising administering to a subject in need thereof a pharmaceutical formulation according to claim 1 in an amount sufficient to mitigate a symptom of Alzheimer's disease.

108. The method of claim 107, wherein said mitigation comprises a reduction of the plaque load in the brain of said subject.
